(19) United States
(12) Patent Application Publication
(10) Pub. No.: US 2024/0189256 A1
(43) Pub. Date: Jun. 13, 2024

(54) COMPOSITIONS FOR PRE-SURGICAL NUTRITIONAL SUPPLEMENTS

(71) Applicant: TM Nutrition LLC, Derby, CT (US)

(72) Inventor: Thea MARX, Derby, CT (US)

(21) Appl. No.: 18/532,735

(22) Filed: Dec. 7, 2023

Related U.S. Application Data
(60) Provisional application No. 63/593,101, filed on Oct. 25, 2023.
(60) Provisional application No. 63/431,092, filed on Dec. 8, 2022.

(51) Int. Cl. A61K 31/07; A61K 9/00; A61K 31/197; A61K 31/198; A61K 31/355; A61K 31/375; A61K 31/4172; A61K 31/59; A61K 31/7016; A61K 31/715; A61K 33/00; A61K 33/06; A61K 33/24; A61K 33/30; A61K 33/32; A61K 33/34; A61P 3/02

(52) U.S. Cl. CPC A61K 31/07; A61K 9/009; A61K 31/197; A61K 31/198; A61K 31/355; A61K 31/375; A61K 31/4172; A61K 31/59; A61K 31/7016; A61K 31/715; A61K 33/00; A61K 33/06; A61K 33/24; A61K 33/30; A61K 33/32; A61K 33/34; A61P 3/02

(57) ABSTRACT

Compositions and methods disclosed herein are configured to be administered to a subject prior to surgery. Compositions and methods disclosed herein are configured to provide nutrition and counteract physiological effects of a surgery. Compositions and methods disclosed herein can enable a subject to mitigate the stress response responsible for drastic shifts in metabolic and biochemical pathways, which can often leave a subject susceptible to a risk of higher mortality.

### CROSS REFERENCE

This application claims the benefit of U.S. Provisional Application No. 63/431,092 filed on Dec. 8, 2022, and U.S. Provisional Application No. 63/593,101 filed on Oct. 25, 2023, which are entirely incorporated herein by reference.

### BACKGROUND

Even the most minor of surgical procedures place acute strain on the patient's body and myriad systems. Patients may find themselves recovering as much from the ancillary effects of their surgical procedures as the particular goals of the procedures themselves, e.g., an excision, replacement, implant, etc., particularly in more invasive procedures.

By way of example, the pain, stress and anesthesia of surgical procedures will increase antioxidant demands, alter the immune system, increase blood glucose, cause cortisol to rise and affect wound healing. The trauma induced by surgery and subsequent wound healing process can increase metabolic needs by 10-100%.

### SUMMARY

In some embodiments, disclosed herein is a composition comprising ingredients, wherein the ingredients comprise, by percent of total mass of the ingredients: i) about 0.00005% w/w to about 0.001% w/w Vitamin A; ii) about 5% w/w to about 15% w/w Vitamin C; iii) about 0.0001% w/w to about 0.01% w/w Vitamin D; iv) about 0.1% w/w to about 10% w/w Vitamin E; v) about 5% to about 15% w/w pantothenic acid; vi) about 0.1% w/w to about 10% w/w calcium; vii) about 0.1% w/w to about 10% w/w magnesium; viii) about 0.01% to about 1% w/w zinc; ix) about 0.001% w/w to about 0.01% w/w selenomethionine; x) about 0.001% w/w to about 0.01% w/w copper; xi) about 0.1% w/w to about 10% w/w potassium; and xii) about 25% w/w to about 75% w/w trehalose.

In some embodiments, disclosed herein is a composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; and xii) 14,000 mg trehalose.

In some embodiments, disclosed herein is a composition comprising: i) 40 μg RAE Vitamin A; ii) 3,000 mg Vitamin C; iii) 60 μg Vitamin D; iv) 450 mg Vitamin E; v) 2,250 mg pantothenic acid; vi) 250 mg calcium; vii) 325 mg magnesium; viii) 28 mg zinc; ix) 600 μg selenomethionine; x) 0.8 mg copper; xi) 325 mg potassium; and xii) 15,000 mg trehalose.

In some embodiments, disclosed herein is a composition comprising: i) 30 μg RAE Vitamin A; ii) 2,000 mg Vitamin C; iii) 75 μg Vitamin D; iv) 400 mg Vitamin E; v) 2,250 mg pantothenic acid; vi) 250 mg calcium; vii) 300 mg magnesium; viii) 35 mg zinc; ix) 500 μg selenomethionine; x) 1 mg copper; xi) 250 mg potassium; and xii) 14,500 mg trehalose.

In some embodiments, disclosed herein is a composition comprising: i) 50 μg RAE Vitamin A; ii) 3,000 mg Vitamin C; iii) 60 μg Vitamin D; iv) 500 mg Vitamin E; v) 2,200 mg pantothenic acid; vi) 230 mg calcium; vii) 375 mg magnesium; viii) 45 mg zinc; ix) 600 μg selenomethionine; x) 0.8 mg copper; xi) 300 mg potassium; and xii) 14,500 mg trehalose.

In some embodiments, disclosed herein is a composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; and xiii) 3,500 mg arabinogalactan.

In some embodiments, disclosed herein is a composition comprising: i) 60 μg RAE Vitamin A; ii) 2,500 mg Vitamin C; iii) 25 μg Vitamin D; iv) 500 mg Vitamin E; v) 2,500 mg pantothenic acid; vi) 210 mg calcium; vii) 400 mg magnesium; viii) 25 mg zinc; ix) 600 μg selenomethionine; x) 1 mg copper; xi) 225 mg potassium; xii) 13,500 mg trehalose; and xiii) 3,000 mg arabinogalactan.

In some embodiments, disclosed herein is a composition comprising: i) 50 μg RAE Vitamin A; ii) 2,250 mg Vitamin C; iii) 40 μg Vitamin D; iv) 300 mg Vitamin E; v) 1,800 mg pantothenic acid; vi) 225 mg calcium; vii) 375 mg magnesium; viii) 40 mg zinc; ix) 500 μg selenomethionine; x) 0.9 mg copper; xi) 200 mg potassium; xii) 13,000 mg trehalose; and xiii) 3,750 mg arabinogalactan.

In some embodiments, disclosed herein is a composition comprising: i) 50 μg RAE Vitamin A; ii) 2,250 mg Vitamin C; iii) 70 μg Vitamin D; iv) 350 mg Vitamin E; v) 1,750 mg pantothenic acid; vi) 175 mg calcium; vii) 325 mg magnesium; viii) 35 mg zinc; ix) 600 μg selenomethionine; x) 0.8 mg copper; xi) 300 mg potassium; xii) 14,500 mg trehalose; and xiii) 4,000 mg arabinogalactan.

In some embodiments, disclosed herein is a composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; xiii) 3,500 mg arabinogalactan; xiv) 3,000 mg L-citrulline; and xv) 300 mg chromium.

In some embodiments, disclosed herein is a composition comprising: i) 40 μg RAE Vitamin A; ii) 3,000 mg Vitamin C; iii) 70 μg Vitamin D; iv) 500 mg Vitamin E; v) 2,250 mg pantothenic acid; vi) 210 mg calcium; vii) 400 mg magnesium; viii) 40 mg zinc; ix) 600 μg selenomethionine; x) 1 mg copper; xi) 300 mg potassium; xii) 13,500 mg trehalose; xiii) 3,500 mg arabinogalactan; xiv) 3,000 mg L-citrulline; and xv) 250 mg chromium.

In some embodiments, disclosed herein is a composition comprising: i) 50 μg RAE Vitamin A; ii) 2,000 mg Vitamin C; iii) 60 μg Vitamin D; iv) 450 mg Vitamin E; v) 2,500 mg pantothenic acid; vi) 250 mg calcium; vii) 375 mg magnesium; viii) 35 mg zinc; ix) 750 μg selenomethionine; x) 0.75 mg copper; xi) 250 mg potassium; xii) 14,000 mg trehalose; xiii) 4,000 mg arabinogalactan; xiv) 2,500 mg L-citrulline; and xv) 250 mg chromium.

In some embodiments, disclosed herein is a composition comprising: i) 40 μg RAE Vitamin A; ii) 2,750 mg Vitamin C; iii) 50 μg Vitamin D; iv) 375 mg Vitamin E; v) 1,800 mg pantothenic acid; vi) 175 mg calcium; vii) 425 mg magnesium; viii) 25 mg zinc; ix) 700 μg selenomethionine; x) 0.7 mg copper; xi) 275 mg potassium; xii) 15,000 mg trehalose; xiii) 3,000 mg arabinogalactan; xiv) 2,500 mg L-citrulline; and xv) 300 mg chromium.

In some embodiments, disclosed herein is a composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; xiii) 3,500 mg arabinogalactan; xiv) 11 mg manganese; and xv) 2,000 mg carnosine.

In some embodiments, disclosed herein is a composition comprising: i) 40 μg RAE Vitamin A; ii) 2,000 mg Vitamin C; iii) 40 μg Vitamin D; iv) 400 mg Vitamin E; v) 2,200 mg pantothenic acid; vi) 250 mg calcium; vii) 350 mg magnesium; viii) 25 mg zinc; ix) 500 μg selenomethionine; x) 0.75 mg copper; xi) 250 mg potassium; xii) 14,000 mg trehalose; xiii) 3,500 mg arabinogalactan; xiv) 9 mg manganese; and xv) 1,500 mg carnosine.

In some embodiments, disclosed herein is a composition comprising: i) 50 μg RAE Vitamin A; ii) 2,000 mg Vitamin C; iii) 60 μg Vitamin D; iv) 450 mg Vitamin E; v) 2,500 mg pantothenic acid; vi) 150 mg calcium; vii) 410 mg magnesium; viii) 35 mg zinc; ix) 500 μg selenomethionine; x) 1 mg copper; xi) 300 mg potassium; xii) 14,500 mg trehalose; xiii) 3,000 mg arabinogalactan; xiv) 10 mg manganese; and xv) 1,750 mg carnosine.

In some embodiments, disclosed herein is a composition comprising: i) 30 μg RAE Vitamin A; ii) 3,000 mg Vitamin C; iii) 50 μg Vitamin D; iv) 425 mg Vitamin E; v) 2,200 mg pantothenic acid; vi) 200 mg calcium; vii) 360 mg magnesium; viii) 40 mg zinc; ix) 350 μg selenomethionine; x) 0.75 mg copper; xi) 250 mg potassium; xii) 15,000 mg trehalose; xiii) 2,750 mg arabinogalactan; xiv) 7.5 mg manganese; and xv) 2,500 mg carnosine.

In some embodiments, disclosed herein is a composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; and xiii) 350 mg gelatin.

In some embodiments, disclosed herein is a composition comprising: i) 50 μg RAE Vitamin A; ii) 2,500 mg Vitamin C; iii) 40 μg Vitamin D; iv) 300 mg Vitamin E; v) 2,500 mg pantothenic acid; vi) 150 mg calcium; vii) 250 mg magnesium; viii) 15 mg zinc; ix) 350 μg selenomethionine; x) 0.5 mg copper; xi) 175 mg potassium; xii) 14,500 mg trehalose; and xiii) 500 mg gelatin.

In some embodiments, disclosed herein is a composition comprising: i) 35 μg RAE Vitamin A; ii) 2,250 mg Vitamin C; iii) 50 μg Vitamin D; iv) 450 mg Vitamin E; v) 2,000 mg pantothenic acid; vi) 200 mg calcium; vii) 350 mg magnesium; viii) 25 mg zinc; ix) 400 μg selenomethionine; x) 0.8 mg copper; xi) 300 mg potassium; xii) 15,000 mg trehalose; and xiii) 400 mg gelatin.

In some embodiments, disclosed herein is a composition comprising: i) 40 μg RAE Vitamin A; ii) 1,850 mg Vitamin C; iii) 60 μg Vitamin D; iv) 500 mg Vitamin E; v) 2,500 mg pantothenic acid; vi) 150 mg calcium; vii) 300 mg magnesium; viii) 40 mg zinc; ix) 550 μg selenomethionine; x) 0.8 mg copper; xi) 250 mg potassium; xii) 14,250 mg trehalose; and xiii) 300 mg gelatin.

In some embodiments, disclosed herein is a method of providing nutrition to a patient prior to surgery, the method comprising: a) preparing a composition including trehalose, arabinogalactan, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, chromium picolinate, and L-citrulline; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

In some embodiments, disclosed herein is a method of providing nutrition to a patient prior to surgery, the method comprising: a) preparing a composition including trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, gelatin, and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

In some embodiments, disclosed herein is a method of providing nutrition to a patient prior to surgery, the method comprising: a) preparing a composition including trehalose, arabinogalactan, carnosine, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, manganese, and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

In some embodiments, disclosed herein is a method of providing nutrition to a patient prior to surgery, the method comprising: a) preparing a composition comprising trehalose, arabinogalactan, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

### BRIEF DESCRIPTION OF THE FIGURES

FIG. 1 illustrates a nutrition label for a sachet formulation comprising vitamin A, vitamin C, vitamin D, vitamin E, panthothenic acid, calcium, magnesium, zinc, selenomethionine, copper, potassium, and trehalose.

FIG. 2 illustrates a nutrition label for a sachet formulation comprising comprising vitamin A, vitamin C, vitamin D, vitamin E, pantothenic acid, calcium, magnesium, zinc, selenomethionine, copper, potassium, trehalose, and arabinogalactan.

FIG. 3 illustrates a nutrition label for a sachet formulation comprising vitamin A, vitamin C, vitamin D, vitamin E, pantothenic acid, calcium, magnesium, zinc, selenomethionine, copper, potassium, trehalose, and arabinogalactan.

FIG. 4 illustrates a nutrition label for a sachet formulation comprising vitamin A, vitamin C, vitamin D, vitamin E, pantothenic acid, calcium, magnesium, zinc, selenomethionine, copper, potassium, trehalose, arabinogalactan, L-citrulline, and chromium.

FIG. 5 illustrates a nutrition label for a sachet formulation comprising Sachet formulations comprising vitamin A, vitamin C, vitamin D, vitamin E, pantothenic acid, calcium, magnesium, zinc, selenomethionine, copper, potassium, trehalose, arabinogalactan, manganese, and carnosine.

FIG. 6 illustrates a nutrition label for a sachet formulation comprising vitamin A, vitamin C, vitamin D, vitamin E, pantothenic acid, calcium, magnesium, zinc, selenomethionine, copper, potassium, trehalose, and gelatin.

### DETAILED DESCRIPTION

Aspects of the present disclosure are directed to compositions administered to patients at predetermined intervals pre-op to counteract the effects of surgical procedures performed on those patients.

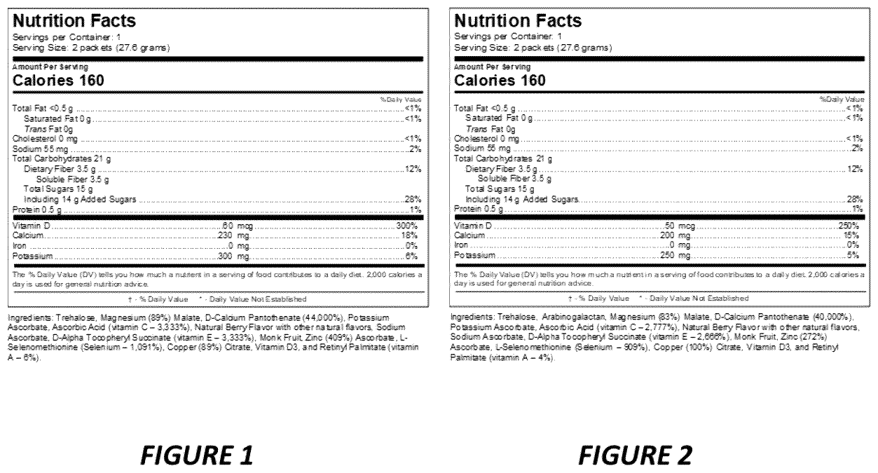

As illustrated in FIG. 1, some embodiments of the present disclosure are directed to a composition configured to be administered to a patient prior to surgery. In some embodiments, a composition of the disclosures is configured to provide nutrition and to counteract physiological effects of the surgery. In some embodiments, a composition of the disclosure is non-hyperglycemic. In some embodiments, a composition of the disclosure is hypoallergenic.

### Nutrients

In some embodiments, a composition of the disclosures comprises trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, and retinyl palmitate, or a functional equivalent thereof.

In some embodiments, a composition of the disclosures comprises trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, and arabinogalactan, or a functional equivalent thereof.

In some embodiments, a composition of the disclosures comprises trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, arabinogalactan, L-citrulline, and chromium or a functional equivalent thereof.

In some embodiments, a composition of the disclosures comprises trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, arabinogalactan, manganese, and carnosine, or a functional equivalent thereof.

In some embodiments, a composition of the disclosures comprises trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, and gelatin, or a functional equivalent thereof.

In some embodiments, a composition of the disclosures further comprises L-arginine, sodium citrate, potassium aspartate, bromelain, manganese, retinoic acid, branch chain amino acids, branched-chain keto acids, leucine, glutamine, ashwagandha (Withania somnifera), Holy Basil, Arnica montana, citric acid, or combinations thereof.

In some embodiments, trehalose is selected for use in a composition of the disclosure, e.g., due to being neuroprotective, a carbohydrate for cellular energy, cellular hydration, antioxidant, and its ability to chaperone protein folding. Trehalose, also known as mycose or tremalose, is a natural alpha-linked disaccharide formed by an 1,1-glucosidic bond between two glucose units. It is naturally found in many animals, plants, and microorganisms including butterflies, grasshoppers, bees and locusts. In these insects, the blood sugar is trehalose. Additionally, trehalose is the molecule that provides these organisms the ability to withstand prolonged droughts and dry spells, known as anhydrobiosis. Trehalose has high water retention capabilities and is often used in cosmetics and food due to that property. In some embodiments, the method of action is that the carbohydrate is thought to form a gel phase as the cells dehydrate. This gel prevents disruption of internal cell organelles by effectively splinting them in position. When rehydration occurs, normal cellular activity can resume without the major and often lethal damage that would typically follow a dehydration/rehydration cycle. As discussed above, trehalose has also found a role in organ protection solutions for organ transplants, taking advantage of its properties to preserve tissue and protein, it has the added advantage of being an antioxidant, and it is being used as a protein-stabilizing agent in emerging research. That protein-stabilizing effect that has been shown to have such benefit in preserving the healthy function of nerves. Under stressful conditions, amyloidogenic proteins undergo an alternative-folding pathway leading to formation of fibrils having cross beta-sheet structure. This is a hallmark of many neurodegenerative diseases. Studies show trehalose prevents this abnormal protein folding and contributes to protein stability, possibly giving way to its efficacy against the amyloid formation associated with neurodegenerative disorders. In some embodiments, trehalose has the ability to improve glucose tolerance by increasing insulin sensitivity and therefore reducing hyperglycemic driven pathology. By inducing transcriptional activation of macrophage autophagy and autophagy-lysosome biosynthesis, the consumption of trehalose can reduce the downstream effects of hyperglycemia, including, but not limited to atherosclerosis formation. Furthermore, the development of arteriosclerosis and aortic aneurysm has been attributed to dysfunctional autophagy in vascular smooth muscle cells because of cell death and aging. By improving arteriosclerosis through the activation of autophagy and an improvement in glucose tolerance, trehalose therefore reduces atherosclerotic risk. Trehalose in a single dose does not stimulate a rapid increase in blood glucose or cause excessive secretion of insulin or gastric inhibitory polypeptide.

In some embodiments, a composition of the disclosure comprises about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, about 10,000 mg, about 10,500 mg, about 11,000 mg, about 11,500 mg, about 12,000 mg, about 12,500 mg, about 13,000 mg, about 13,500 mg, about 14,000 mg, about 14,500 mg, about 15,000 mg, about 15,500 mg, about 16,000 mg, about 16,500 mg, about 17,000 mg, about 17,500 mg, about 18,000 mg, about 18,500 mg, about 19,000 mg, about 19,500 mg, or about 20,000 mg of trehalose or a functional equivalent thereof. In some embodiments, a composition of the disclosure can comprise about 10,000 mg of trehalose or a functional equivalent thereof. In some embodiments, a composition of the disclosure can comprise about 12,500 mg of trehalose or a functional equivalent thereof. In some embodiments, a composition of the disclosure can comprise about 14,000 mg of trehalose or a functional equivalent thereof. In some embodiments, a composition of the disclosure can comprise about 16,000 mg of trehalose or a functional equivalent thereof.

In some embodiments, a composition of the disclosure can comprise at least about 1% w/w, at least about 5% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of trehalose or a functional equivalent thereof. In some embodiments, a composition of the disclosure can comprise at least about 25% w/w of trehalose or a functional equivalent thereof. In some embodiments, a composition of the disclosure can comprise at least about 50% w/w of trehalose or a functional equivalent thereof. In some embodiments, a composition of the disclosure can comprise at least about 75% of trehalose or a functional equivalent thereof. In some embodiments, a composition of the disclosure can comprise between about 5% w/w to about 15% w/w, between about 15% w/w to about 25% w/w, between about 25% w/w to about 50% w/w, between about 25% w/w to about 75% w/w, between about 50% to about 75% w/w trehalose or a functional equivalent thereof. In some embodiments, a composition of the disclosure can comprise between about 25% to about 75% w/w trehalose or a functional equivalent thereof.

In some embodiments, arabinogalactan is selected for use in a composition of the disclosure, e.g., due to being advantageous for immune support and protection of the microbiome. High-molecular weight polysaccharides from the western larch tree have the ability to up-regulate the immune system. It is a good source of soluble fiber that promotes colon health. Arabinogalactans are a class of polysaccharides found in many plants. They are most abundant in plants of the genus Larix (larch tree is Larix occidentalis). High-grade or nutraceutical-grade Larch Arabinogalactan (the grade most often utilized for supplements) is composed of greater than 98% arabinogalactan. Larch Arabinogalactan is a dry, free-flowing powder, with a very gentle pine-like odor and slightly sweet taste. It is 100% water-soluble and produces low viscosity solutions. Due to its excellent solubility and mild taste, the powder mixes readily in juices and water. It is easily administered to patients including children and elderly. Larch arabinogalactan is a source of dietary fiber. Research shows it to increase the production of short-chain fatty acids (SCFAs), principally butyrate and propionate. These special fatty acids are beneficial for colon health. An adequate supply of SCFA's appears to make colon cells more resistant to both a number of intestinal diseases as well as tumor promotion. Larch arabinogalactan can also act as a prebiotic for commensal bacteria. A non-limiting example of a prebiotic substance is “fructo-oligosaccharides” or “FOS.” In humans, larch arabinogalactan acts in the same manner as FOS. In effect, when consumed, larch arabinogalactan has a significant positive effect on the gut microbiome balance. In some embodiments, this fiber acts by increasing good bacteria like Bifidobacteria and Lactobacillus, while decreasing bad bacteria. These friendly bacteria improve the health of our digestive and immune systems, hormone regulating capabilities, detoxification, and nutrient formation and absorption. Research has shown that arabinogalactan appears to enhance natural killer cell cytotoxicity and macrophage activation. It also plays a role in blocking organ-specific metastasis.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg of arabinogalactan or a functional equivalent thereof. In some embodiments, a composition of the disclosure can comprise about 2,000 mg of arabinogalactan or a functional equivalent thereof. In some embodiments, a composition of the disclosure can comprise about 3,500 mg of arabinogalactan or a functional equivalent thereof. In some embodiments, a composition of the disclosure can comprise about 5,000 mg of arabinogalactan or a functional equivalent thereof. In some embodiments, a composition of the disclosure can comprise about 7,500 mg of arabinogalactan or a functional equivalent thereof.

In some embodiments, a composition of the disclosure can comprise at least about 1% w/w, at least about 5% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of arabinogalactan or a functional equivalent thereof. In some embodiments, a composition of the disclosure can comprise at least about 5% w/w of arabinogalactan or a functional equivalent thereof. In some embodiments, a composition of the disclosure can comprise at least about 10% w/w of arabinogalactan or a functional equivalent thereof. In some embodiments, a composition of the disclosure can comprise at least about 15% w/w of arabinogalactan or a functional equivalent thereof. In some embodiments, a composition of the disclosure can comprise at least about 20% w/w of arabinogalactan or a functional equivalent thereof. In some embodiments, a composition of the disclosure can comprise at least about 25% w/w of arabinogalactan or a functional equivalent thereof. In some embodiments, a composition of the disclosure can comprise between about 5% w/w to about 10% w/w, between about 5% w/w to about 15% w/w, between about 5% w/w to about 20% w/w, between about 5% w/w to about 25% w/w, between about 10% w/w to about 15% w/w, between about 10% to about 20% w/w, between about 10% w/w to 25% w/w of arabinogalactan or a functional equivalent thereof. In some embodiments, a composition of the disclosure can comprise between about 5% w/w to about 25% w/w arabinogalactan or a functional equivalent thereof.

In some embodiments, Vitamin C and its derivatives, e.g., potassium ascorbate, sodium ascorbate, zinc ascorbate, ascorbic acid, etc., are selected for use in embodiments of a composition of the disclosure, e.g., for tissue growth and repair and its role in formation of collagen for wound healing. If deficient, water soluble vitamin C will contribute to fragile granulation tissue. Adequate levels of vitamin C affect the function of the enzyme protocollagen hydroxylase, which produces collagen, the primary constituent of granulation tissue. Vitamin C is a component of the wound-healing process. There is clinical evidence that wound healing benefits from more vitamin than can be achieved with diet alone. Daily replenishment through supplements is beneficial because vitamin C is water-soluble, ensuring excess is excreted not stored. Ophthalmologists routinely administer vitamin C to patients undergoing corneal transplantation where wound healing is closely monitored.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg vitamin C, its derivatives, or a functional equivalent thereof. In some embodiments, a composition of the disclosure can comprise about 1,000 mg of vitamin C, its derivatives, or a functional equivalent thereof. In some embodiments, a composition of the disclosure can comprise about 2,000 mg of vitamin C, its derivatives, or a functional equivalent thereof. In some embodiments, a composition of the disclosure can comprise about 2,500 mg of vitamin C, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of vitamin C, its derivatives, or a functional equivalent thereof. In some embodiments, a composition of the disclosure can comprise at least about 2.5% w/w vitamin C, its derivatives or a functional equivalent thereof. In some embodiments, a composition of the disclosure can comprise at least about 5.0% w/w vitamin C, its derivatives or a functional equivalent thereof. In some embodiments, a composition of the disclosure can comprise at least about 7.5% w/w vitamin C, its derivatives or a functional equivalent thereof. In some embodiments, a composition of the disclosure can comprise at least about 9.0% w/w vitamin C, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w vitamin C, its derivatives, or function equivalents thereof. In some embodiments, a composition of the disclosure can comprise between about 5% w/w to about 15% w/w vitamin C, its derivatives, or a functional equivalent thereof.

In some embodiments, D-alpha tocopheryl succinate is selected for use in embodiments of a composition of the disclosure, e.g., to metabolize free radicals, decreasing the risk of disease, as well as reducing lipid peroxidation, thrombosis and platelet adhesiveness. Released from the liver in association with LDL, vitamin E (as systemic alpha tocopherols) circulates in the blood bound most often to very low-density lipoproteins. Its delivery and concentration to extrahepatic tissues are seemingly regulated by factors controlling low-density lipoprotein carrier capacity and total lipid content of plasma. Secondary catabolic stress depletion of transport capacity affects plasma levels of both vitamin E and A. Benefits of fat soluble vitamins arise from a complex antioxidant defense system in the human body which is activated to prevent disintegration of tissues. The surgical process generates oxygen derived free-radicals that are responsible for many complications including myocardial stunning or hibernation—a reduction in cardiac performance, specifically impaired myocardial contractility and decreased left ventricular function persisting several days or longer after reperfusion. A complementary strategy has been proposed to support the body's antioxidant defense system due to the impaired secretion of VLDL and retinol-binding protein (RBP) from the liver in patients undergoing surgery, potentially a result of insufficient liver perfusion and hypothermia causing decreased metabolic activity. Vitamin A decreased significantly, presumably used by the tissues to combat oxidative stress when acute phase response depresses RBP, thus its decline is due to reduced synthesis of its carrier protein. Vitamin E plasma levels are in equilibrium to the stores in tissues. Low circulating levels of alpha tocopherol may be an indication that reserves in the heart are decreasing, leaving patients more susceptible to oxidative injury. In some embodiments, it would be beneficial for patients to receive perioperative vitamin E in doses large enough to saturate circulating carrier lipoprotein molecules, but low enough not to induce anti-coagulant effects. For vitamin A, circumventing the use of RBP and supplementing with retinoic acid, which is carried by albumin, would potentially keep levels elevated in the plasma. The body has a defense network of antioxidants that provide protection in an effort to prevent body tissues from disintegrating and are replenished to continue the healing process. Especially potent is the antioxidant vitamin E which metabolizes free radicals.

In some embodiments, a composition of the disclosure can comprise about 10 mg, about 20 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,250 mg, about 1,500 mg, or about 2,000 mg Vitamin E, its derivatives, or a functional equivalent thereof (e.g., D-alpha tocopheryl succinate). In some embodiments, a composition of the disclosure can comprise about 200 mg of vitamin E, its derivatives, or a functional equivalent thereof. In some embodiments, a composition of the disclosure can comprise about 400 mg of vitamin E, its derivatives, or a functional equivalent thereof. In some embodiments, a composition of the disclosure can comprise about 600 mg of vitamin E, its derivatives, or a functional equivalent thereof.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w vitamin E, its derivatives, or a functional equivalent thereof. In some embodiments, a composition of the disclosure can comprise at least about 1.0% w/w vitamin E, its derivatives, or a functional equivalent thereof. In some embodiments, a composition of the disclosure can comprise at least about 1.5% w/w vitamin E, its derivatives, or a functional equivalent thereof. In some embodiments, a composition of the disclosure can comprise at least about 2.0% w/w vitamin E, its derivatives, or a functional equivalent thereof. In some embodiments, a composition of the disclosure can comprise at least about 3.0% w/w vitamin E, its derivatives, or a functional equivalent thereof. In some embodiments, a composition of the disclosure can comprise between about 0.5% w/w to 1.5% w/w, between about 1.0% w/w to about 5% w/w, between about 1.0% w/w to about 10% w/w, or between about 5% to about 10% w/w vitamin E, its derivatives, or a functional equivalent thereof.

In some embodiments, vitamin D3 is selected for use in embodiments of a composition of the disclosure, e.g., to regulate the inflammatory response from the patient. Vitamin D receptors are found in most body tissues and cells. 1,25(OH) D regulates approximately 200 genes such as rennin in the kidney, insulin production in the pancreas, cytokine release from lymphocytes, enhances cathelicidin production. Vitamin D reduces the development of atherosclerosis by inhibiting monocyte/macrophage pro-inflammatory cytokine production. It also positively influences vascular smooth muscle cells (vSMCs) helping them maintain their integrity, normal proliferation and prevent migration therefore reducing the development of the diabetic atherosclerotic process. Vascular smooth muscle cells and monocytes/macrophages and) are the main cell types implicated in atherosclerosis development. Unlike other mature cell types, both retain a significant plasticity. Differentiated vSMCs, in mature vessels, control blood pressure and the vascular tone. Responding to modifications of the local environment (inflammation, oxidative stress) and vascular injury vSMCs switch from a contractile to a secretory phenotype as well as display macrophagic markers expression and a macrophagic behavior increasing potential formation of atherosclerosis. Vitamin D has cardiovascular pleiotropic effects by activating nuclear VDR (Vitamin D Receptors) in vascular endothelial cells and cardiomyocytes and regulating the renin-angiotensin-aldosterone system, energy expense, adiposity, and pancreatic cell activity. It increases the absorption of calcium from the duodenum and phosphorus from the ileum. Vitamin D increases bone resorption, decreases parathyroid hormone synthesis and secretion, increases insulin production, decreases the synthesis of rennin and improves myocardial contractility. It regulates endothelial functions, stimulates nitric oxide production and reduces oxidative stress. Vitamin D deficiency may be effective in insulin resistance and type 2 DM pathogenesis by affecting both β-cell function and insulin sensitivity. It is involved in the immune response as Vitamin D favors a Th2 response, suppressing IL-2, TNF-alpha and INF-gamma while increasing IL-4, IL-5 and IL-10 as well as suppressing dendritic cells.

In some embodiments, a composition of the disclosure can comprise about 10 μg, about 20 μg, about 25 μg, about 30 μg, about 35 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 750 μg, or about 1,000 μg Vitamin D, its derivatives, or a functional equivalent thereof. In some embodiments, a composition of the disclosure can comprise about 25 μg vitamin D, its derivatives, or a functional equivalent thereof. In some embodiments, a composition of the disclosure can comprise about 50 μg vitamin D, its derivatives or a functional equivalent thereof. In some embodiments, a composition of the disclosure can comprise about 75 μg vitamin D, its derivatives, or a functional equivalent thereof. In some embodiments, a composition of the disclosure can comprise about 100 μg vitamin D, its derivatives, or a functional equivalent thereof.

In some embodiments, a composition of the disclosure can comprise at least about 0.00005% w/w, at least about 0.0001% w/w, at least about 0.00015% w/w, at least about 0.0002% w/w, at least about 0.00025% w/w, at least about 0.0003%, at least about 0.00035% w/w, at least about 0.0004% w/w, at least about 0.00045% w/w, at least about 0.0005%, at least about 0.0006% w/w, at least about 0.0007% w/w, at least about 0.0008% w/w, at least about 0.0009% w/w, at least about 0.001%, at least about 0.002%, at least about 0.0025%, at least about 0.005% w/w, at least about 0.0075% w/w, at least about 0.01% w/w, at least about 0.025%, at least about 0.05%, or at least about 0.1% w/w vitamin D, its derivatives, or a functional equivalent thereof. In some embodiments, a composition of the disclosure can comprise at least about 0.001% w/w vitamin D, its derivatives, or a functional equivalent thereof. In some embodiments, a composition of the disclosure can comprise at least about 0.002% w/w vitamin D, its derivatives, or a functional equivalent thereof. In some embodiments, a composition of the disclosure can comprise at least about 0.0025% w/w vitamin D, its derivatives, or a functional equivalent thereof. In some embodiments, a composition of the disclosure can comprise at least about 0.005% w/w vitamin D, its derivatives, or a functional equivalent thereof. In some embodiments, a composition of the disclosure can comprise between about 0.0001% w/w to about 0.0005% w/w, between about 0.0001% w/w to about 0.00075% w/w, between about 0.0001% w/w to about 0.001% w/w, between about 0.0001% w/w to about 0.005% w/w, or between about 0.0001% w/w to about 0.01% w/w vitamin D, its equivalents, or functional derivatives thereof.

In some embodiments, Vitamin B and its derivatives, e.g., pantothenic acid, D-calcium pantothenate, etc., is selected for use in embodiments of a composition of the disclosure, e.g., to blunt overactive cortisol due to stress. Pantothenic acid is one of the 8 B-vitamins. B5 is water-soluble and it is found in every cell in the body. Pantothenic acid can induce adrenal hyperresponsiveness to ACTH stimulation, and PRL further stimulates adrenal sensitivity to ACTH. Pantothenic acid is a constituent of Coenzyme A which is used in the synthesis of acetylcholine, serotonin and epinephrine. Acetylcholine in adequate levels can boost memory, focus, learning, and reduce brain fog, as well as convert nutrients from food into energy through the Krebs cycle and electron transport chain which is used to make ATP (adenosine triphosphate). B5 helps lower blood pressure, balance blood sugar, reduce LDL, prevent nerve pain and damage, as well as prevent heart failure. Vitamin B5 is involved in the synthesis and metabolism of carbohydrates, proteins and fats. It converts these macronutrients into energy that the brain uses to fuel neurotransmitters. It is also utilized in the manufacture of red blood cells as well as stress and sex hormones produced in the adrenal glands. Adrenal glands use CoA together with cholesterol and Vitamin C to manufacture epinephrine and cortisol. In doing so it can reduce stress, anxiety and depression.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg pantothenic acid, its derivatives, or a functional equivalent thereof. In some embodiments, a composition of the disclosure can comprise about 1,000 mg pantothenic acid, its derivatives, or a functional equivalent thereof. In some embodiments, a composition of the disclosure can comprise about 2,000 mg pantothenic acid, its derivatives, or a functional equivalent thereof. In some embodiments, a composition of the disclosure can comprise about 4,000 mg pantothenic acid, its derivatives, or a functional equivalent thereof. In some embodiments, a composition of the disclosure can comprise about 6,000 mg pantothenic acid, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w pantothenic acid, its derivatives, or a functional equivalent thereof. In some embodiments, a composition of the disclosure can comprise at least about 2.5% w/w pantothenic acid its derivatives or a functional equivalent thereof. In some embodiments, a composition of the disclosure can comprise at least about 5.0% w/w pantothenic acid, its derivatives or a functional equivalent thereof. In some embodiments, a composition of the disclosure can comprise at least about 7.5% w/w pantothenic acid, its derivatives or a functional equivalent thereof. In some embodiments, a composition of the disclosure can comprise at least about 9.0% w/w pantothenic acid, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w pantothenic acid, its derivatives, or function equivalents thereof. In some embodiments, a composition of the disclosure can comprise between about 5% w/w to about 15% w/w pantothenic acid, its derivatives, or a functional equivalent thereof.

In some embodiments, Vitamin A and its derivatives, e.g., retinyl palmitate, retinoic acid, etc., are antioxidants selected for use in embodiments of a composition of the disclosure, e.g., to aid new cell growth and maintenance and repair of epithelial tissue. Vitamin A is released at a steady rate into the plasma to meet the demands of the liver (where over 90% of retinoid reserves in body are stored). Concentration of retinol is secreted into systemic circulation in a tightly regulated process bound to a retinol binding protein. Vitamin A also helps regulate the immune system. It is involved in lymphopoiesis, cytokine expression, antibody production, and the function of nearly all white blood cells. Natural killer cells, macrophages, and lymphocytes are activated by vitamin A.

In some embodiments, a composition of the disclosure can comprise about 10 μg, about 20 μg, about 25 μg, about 30 μg, about 35 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 750 μg, or about 1,000 μg RAE vitamin A, its derivatives, or a functional equivalent thereof. In some embodiments, a composition of the disclosure can comprise about 10 μg retinol activity equivalents RAE vitamin A, its derivatives, or a functional equivalent thereof. In some embodiments, a composition of the disclosure can comprise about 20 μg RAE vitamin A, its derivatives or a functional equivalent thereof. In some embodiments, a composition of the disclosure can comprise about 40 μg RAE vitamin A, its derivatives, or a functional equivalent thereof. In some embodiments, a composition of the disclosure can comprise about 80 μg RAE vitamin A, its derivatives, or a functional equivalent thereof.

In some embodiments, a composition of the disclosure can comprise at least about 0.00005% w/w, at least about 0.0001% w/w, at least about 0.00015% w/w, at least about 0.0002% w/w, at least about 0.00025% w/w, at least about 0.0003%, at least about 0.00035% w/w, at least about 0.0004% w/w, at least about 0.00045% w/w, at least about 0.0005%, at least about 0.0006% w/w, at least about 0.0007% w/w, at least about 0.0008% w/w, at least about 0.0009% w/w, at least about 0.001%, at least about 0.002%, at least about 0.0025%, at least about 0.005% w/w, at least about 0.0075% w/w, at least about 0.01% w/w, at least about 0.025%, at least about 0.05%, or at least about 0.1% w/w RAE vitamin A, its derivatives, or a functional equivalent thereof. In some embodiments, a composition of the disclosure can comprise at least about 0.0001% w/w RAE vitamin A, its derivatives, or a functional equivalent thereof. In some embodiments, a composition of the disclosure can comprise at least about 0.0002% w/w RAE vitamin A, its derivatives, or a functional equivalent thereof. In some embodiments, a composition of the disclosure can comprise at least about 0.00025% w/w RAE vitamin A, its derivatives, or a functional equivalent thereof. In some embodiments, a composition of the disclosure can comprise at least about 0.0005% w/w RAE vitamin A, its derivatives, or a functional equivalent thereof. In some embodiments, a composition of the disclosure can comprise between about 0.00005% w/w to about 0.0001% w/w, between about 0.00005% w/w to about 0.0005% w/w, between about 0.00005% w/w to about 0.001% w/w, between about 0.00005% w/w to about 0.005% w/w, or between about 0.00005% w/w to about 0.01% w/w RAE vitamin A, its equivalents, or functional derivatives thereof.

In some embodiments, a composition of the disclosure can comprise calcium pantothenate as a calcium source. In some embodiments, a composition of the disclosure can comprise about 10 mg, about 25 mg, about 50 mg, about 75 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 450 mg, about 500 mg, about 600 mg, about 700 mg, about 800 mg, about 900 mg, or about 1,000 mg of the calcium cation (Ca²⁺). In some embodiments, a composition of the disclosure can comprise about 100 mg Ca²⁺. In some embodiments, a composition of the disclosure can comprise about 200 mg Ca²⁺. In some embodiments, a composition of the disclosure can comprise about 400 mg Ca²⁺. In some embodiments, a composition of the disclosure can comprise about 500 mg Ca²⁺.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w Ca²⁺. In some embodiments, a composition of the disclosure can comprise at least about 0.5% w/w Ca²⁺. In some embodiments, a composition of the disclosure can comprise at least about 0.7% w/w Ca²⁺. In some embodiments, a composition of the disclosure can comprise at least about 1.0% w/w Ca²⁺. In some embodiments, a composition of the disclosure can comprise at least about 2.0% w/w Ca²⁺. In some embodiments, a composition of the disclosure can comprise at least about 3.0% w/w Ca²⁺. In some embodiments, a composition of the disclosure can comprise between about 0.1% w/w to about 0.5% w/w, between about 0.1% to about 1.0% w/w, between about 0.1% w/w to about 5.0% w/w, between about 0.1% w/w to about 10.0% w/w Ca²⁺.

In some embodiments, sodium is selected for use in embodiments of a composition of the disclosure, e.g., to regulate extracellular fluid and plasma volume, and aid in nerve impulse conduction and muscle contraction control. Sodium is a cation found in extracellular fluid (the dissolved substances and water in the spaces outside cells). In some embodiments, a composition of the disclosure comprises sodium or the sodium cation (Na). Non-limiting examples of sodium sources suitable for use in the composition include: sodium citrate, sodium ascorbate, and sodium chloride.

In some embodiments, a composition of the disclosure can comprise about 10 mg, about 25 mg, about 50 mg, about 75 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 450 mg, about 500 mg, about 600 mg, about 700 mg, about 800 mg, about 900 mg, or about 1,000 mg Na⁺. In some embodiments, a composition of the disclosure can comprise about 100 mg Na⁺. In some embodiments, a composition of the disclosure can comprise about 200 mg Na⁺. In some embodiments, a composition of the disclosure can comprise about 400 mg Na⁺. In some embodiments, a composition of the disclosure can comprise about 500 mg Na⁺.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w Na⁺. In some embodiments, a composition of the disclosure can comprise at least about 0.5% w/w Na⁺. In some embodiments, a composition of the disclosure can comprise at least about 0.7% w/w Na⁺. In some embodiments, a composition of the disclosure can comprise at least about 1.0% w/w Na⁺. In some embodiments, a composition of the disclosure can comprise at least about 2.0% w/w Na⁺. In some embodiments, a composition of the disclosure can comprise at least about 3.0% w/w Na⁺. In some embodiments, a composition of the disclosure can comprise between about 0.1% w/w to about 0.5% w/w, between about 0.1% to about 1.0% w/w, between about 0.1% w/w to about 5.0% w/w, between about 0.1% w/w to about 10.0% w/w Na⁺.

In some embodiments, a composition of the disclosure comprises potassium or the potassium cation (K⁺). Non-limiting examples of a potassium source suitable for use in the composition include: potassium aspartate, potassium ascorbate, and potassium chloride. Potassium works with sodium to maintain a normal water balance, acid base balance, and osmotic equilibrium. It is utilized to regulate neuromuscular activity. Potassium helps prevent digitalis toxicity, nausea, vomiting, abdominal discomfort, cardiac arrythmias, and can promote cellular growth.

In some embodiments, a composition of the disclosure can comprise about 10 mg, about 25 mg, about 50 mg, about 75 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 450 mg, about 500 mg, about 600 mg, about 700 mg, about 800 mg, about 900 mg, or about 1,000 mg K⁺. In some embodiments, a composition of the disclosure can comprise about 100 mg K⁺. In some embodiments, a composition of the disclosure can comprise about 250 mg K⁺. In some embodiments, a composition of the disclosure can comprise about 500 mg K⁺. In some embodiments, a composition of the disclosure can comprise about 1000 mg K⁺.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w K⁺. In some embodiments, a composition of the disclosure can comprise at least about 0.3% w/w K⁺. In some embodiments, a composition of the disclosure can comprise at least about 0.6% w/w K⁺. In some embodiments, a composition of the disclosure can comprise at least about 0.9% w/w K⁺. In some embodiments, a composition of the disclosure can comprise at least about 1.2% w/w K⁺. In some embodiments, a composition of the disclosure can comprise at least about 2.0% w/w K⁺. In some embodiments, a composition of the disclosure can comprise between about 0.1% w/w to about 0.5% w/w, between about 0.1% to about 1.0% w/w, between about 0.1% w/w to about 5.0% w/w, between about 0.1% w/w to about 10.0% w/w K.

In some embodiments, copper, e.g., copper citrate, etc., is selected for use in embodiments of a composition of the disclosure, e.g., due to being a constituent in normal blood and established essential micronutrient. It is a component in many enzymes. It is utilized for cross-linking of collagen and elastin; for formation of hemoglobin, red blood cells, and bone. It is utilized for connective tissue proteins of high tensile strength. It also plays a role in mitochondrial energy production, protection against oxidants and free radicals. It is also utilized for the synthesis of melatonin and catecholamines.

In some embodiments, a composition of the disclosure can comprise about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 3.0 mg, about 4.0 mg, about 5.0 mg, about 6.0 mg, about 7.0 mg, about 8.0 mg, about 9.0 mg, or about 10.0 mg cationic copper. In some embodiments a composition of the disclosure can comprise about 0.3 mg cationic copper. In some embodiments, a composition of the disclosure can comprise about 0.6 mg cationic copper. In some embodiments, a composition of the disclosure can comprise about 0.9 mg cationic copper. In some embodiments, a composition of the disclosure can comprise about 1.2 mg cationic copper.

In some embodiments, a composition of the disclosure can comprise at least about 0.001% w/w, at least about 0.0015% w/w, at least about 0.002% w/w, at least about 0.0025% w/w, at least about 0.003% w/w, at least about 0.0035% w/w, at least about 0.004% w/w, at least about 0.0045% w/w, at least about 0.005% w/w, at least about 0.0055% w/w, at least about 0.006% w/w, at least about 0.0065% w/w, at least about 0.007% w/w, at least about 0.0075% w/w, at least about 0.008% w/w, at least about 0.0085% w/w, at least about 0.009% w/w, at least about 0.0095% w/w, or at least at least about 0.01% w/w copper. In some embodiments, a composition of the disclosure can comprise at least about 0.0015% w/w copper. In some embodiments, a composition of the disclosure can comprise at least about 0.003% w/w copper. In some embodiments, a composition of the disclosure can comprise at least about 0.0045% w/w copper. In some embodiments, a composition of the disclosure can comprise at least about 0.006% w/w copper. In some embodiments, a composition of the disclosure can comprise between about 0.001% w/w to about 0.005% w/w, between about 0.001% w/w to about 0.0075% w/w, between about 0.001% w/w to about 0.01% w/w, or between about 0.005% w/w to about 0.01% w/w copper.

In some embodiments, zinc is selected for use in embodiments of a composition of the disclosure, e.g., due to playing a vital role in more than 300 enzymes that facilitate chemical reactions for immune function. Zinc is an antioxidant that mitigates damage done by the innate immune system to host cells. It is also a component of the function of macrophages, neutrophils and NK cell activity and the Th1 response reducing IFN-gamma and IL-2. Along with vitamin A, it contributes to the integrity of the mucosal barrier. Zinc is utilized for protein synthesis and collage formation and for development and activation of T-lymphocytes. Even a moderate deficiency can adversely affect the immune system. Zinc deficiency can manifest an increased susceptibility to a variety of pathogens through multiple pathways, from the barrier of the skin to gene regulation within lymphocytes. Zinc affects immunologic mediators due to its role in basic cell functions such as DNA replication, RNA transcription, cell division, and cell activation. Small doses of zinc supplements can increase T-lymphocyte levels and have the potential to decrease the incidence of postsurgical infection and its associated complications. Adequate zinc status has a direct impact on the cascade for tissue utilization of vitamin A including metabolism, absorption and hepatic release. Zinc is utilized for synthesis of RBP for the inter and intracellular transport of vitamin A as well as lymphatic absorption of retinol. Conversely, a vitamin A deficiency causes a reduction in zinc-binding protein and thus zinc absorption.

In some embodiments, a composition of the disclosure comprises zinc or a cationic form of zinc (Zn²⁺). Non-limiting examples of zinc sources suitable for use in a composition of the disclosure include zinc citrate and zinc chloride. In some embodiments, a composition of the disclosure can comprise about 1 mg, about 5 mg, about 7.5 mg, about 10 mg, about 12.5 mg, about 15 mg, about 17.5 mg, about 20.0 mg, about 22.5 mg, about 25.0 mg, about 27.5 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, or about 500 mg cationic zinc. In some embodiments, a composition of the disclosures comprises about 10 mg cationic zinc. In some embodiments, a composition of the disclosure can comprise about 20 mg cationic zinc. In some embodiments, a composition of the disclosure can comprise about 30 mg cationic zinc. In some embodiments, a composition of the disclosure can comprise about 50 mg cationic zinc. In some embodiments, a composition of the disclosure can comprise about 100 mg cationic zinc.

In some embodiments, a composition of the disclosure can comprise at least about 0.01% w/w, at least about 0.015% w/w, at least about 0.02% w/w, at least about 0.025% w/w, at least about 0.03% w/w, at least about 0.035% w/w, at least about 0.04% w/w, at least about 0.045% w/w, at least about 0.05% w/w, at least about 0.055% w/w, at least about 0.06% w/w, at least about 0.065% w/w, at least about 0.07% w/w, at least about 0.075% w/w, at least about 0.08% w/w, at least about 0.085% w/w, at least about 0.09% w/w, at least about 0.095% w/w, at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, or at least about 1.0% w/w cationic zinc. In some embodiments, a composition of the disclosure can comprise at least about 0.05% w/w cationic zinc. In some embodiments, a composition of the disclosure can comprise at least about 0.1% w/w cationic zinc. In some embodiments, a composition of the disclosure can comprise at least about 0.2% w/w cationic zinc. In some embodiments, a composition of the disclosure can comprise at least about 0.5% w/w zinc. In some embodiments, a composition of the disclosure can comprise at least about 1.0% w/w cationic zinc. In some embodiments, a composition of the disclosure can comprise between about 0.01% w/w to about 0.05% w/w, between about 0.01% w/w to about 0.075% w/w, between about 0.01% to about 0.1% w/w, between about 0.01% w/w to about 0.5% w/w, or between about 0.01% w/w to about 1.0% w/w cationic zinc.

In some embodiments, manganese is selected for use in embodiments of a composition of the disclosure, e.g., to aid in the metabolism of glucose, cholesterol, amino acids and carbohydrates as well as scavenging reactive oxygen species, bone formation, immune response and reproduction. Manganese is a co-factor for many enzymes including Manganese SOD (Mn-SOD), an inducible antioxidant found locally in the mitochondria, pyruvate carboxylase and arginase. Manganese plays a part in clotting and hemostasis along with Vitamin K. Absorption efficiency increases with low intake of manganese. Men absorb it less efficiently than women, possibly due to iron status which has an inverse relationship to manganese. Iron deficiency increases absorption of manganese. In some embodiments, tolerable upper intake for adults is about 11 mg. Non-limiting examples of a manganese source suitable for use in a composition of the disclosure include manganese ascorbate, manganese aspartate, manganese citrate, manganese picolinate, manganese glycinate, manganese gluconate, or other chelated manganese salts, manganese dioxide, manganese carbonate, and manganese sulfate.

In some embodiments, a composition of the disclosure can comprise about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 3.0 mg, about 4.0 mg, about 5.0 mg, about 6.0 mg, about 7.0 mg, about 8.0 mg, about 9.0 mg, about 10.0 mg manganese, or about 11.0 mg cationic manganese (Mg²⁺). In some embodiments a composition of the disclosure can comprise about 0.3 mg Mg²⁺. In some embodiment, a composition of the disclosure can comprise about 0.6 mg Mg²⁺. In some embodiments, a composition of the disclosure can comprise about 0.9 mg Mg²⁺. In some embodiments, a composition of the disclosure can comprise about 1.2 mg Mg²⁺. In some embodiments, a composition of the disclosure can comprise about 2.4 mg Mg²⁺. In some embodiments, a composition of the disclosure can comprise about 5.0 mg Mg²⁺. In some embodiments, a composition of the disclosure can comprise about 11 mg Mg²⁺.

In some embodiments, a composition of the disclosure can comprise at least about 0.001% w/w, at least about 0.0015% w/w, at least about 0.002% w/w, at least about 0.0025% w/w, at least about 0.003% w/w, at least about 0.0035% w/w, at least about 0.004% w/w, at least about 0.0045% w/w, at least about 0.005% w/w, at least about 0.0055% w/w, at least about 0.006% w/w, at least about 0.0065% w/w, at least about 0.007% w/w, at least about 0.0075% w/w, at least about 0.008% w/w, at least about 0.0085% w/w, at least about 0.009% w/w, at least about 0.0095% w/w, or at least about 0.01% w/w Mg²⁺. In some embodiments, a composition of the disclosure can comprise at least about 0.0015% w/w Mg²⁺. In some embodiments, a composition of the disclosure can comprise at least about 0.003% w/w Mg²⁺. In some embodiments, a composition of the disclosure can comprise at least about 0.0045% w/w Mg²⁺. In some embodiments, a composition of the disclosure can comprise at least about 0.006% w/w Mg²⁺. In some embodiments, a composition of the disclosure can comprise between about 0.001% w/w to about 0.005% w/w, between about 0.001% w/w to about 0.0075% w/w, between about 0.001% w/w to about 0.01% w/w, or between about 0.005% w/w to about 0.01% w/w Mg²⁺.

In some embodiments, chromium or cationic chromium (Cr³⁺) is selected for use in embodiments of a composition of the disclosure, e.g., to potentiate the action of insulin therefore influencing lipid, carbohydrate and protein metabolism. Non-limiting examples of a chromium source suitable for use in a composition of the disclosure include chromium picolinate, chromium-enriched years, chromium polynicotinate, chromium chloride, and chromium histidinate. It also plays a role in regulation of gene expression. It is a modulator of cortisol/HPA axis. Chromium plays a part in glucose tolerance factor (GTF), an essential activator of insulin-mediated reactions. It helps to maintain normal glucose metabolism and peripheral nerve function. Chromium increases insulin binding to cells, increases insulin receptor density, and activates insulin receptor kinase leading to enhanced insulin sensitivity.

In some embodiments, a composition of the disclosure can comprise about 10 μg, about 20 μg, about 25 μg, about 30 μg, about 35 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 750 μg, or about 1,000 μg chromium picolinate equivalents, derivatives, or a functional equivalent thereof.

In some embodiments, a composition of the disclosure can comprise about 150 μg chromium picolinate equivalents, derivatives, or a functional equivalent thereof. In some embodiments, a composition of the disclosure can comprise about 300 μg chromium picolinate equivalents, derivatives, or a functional equivalent thereof. In some embodiments, a composition of the disclosure can comprise about 350 μg chromium picolinate equivalents, derivatives, or a functional equivalent thereof. In some embodiments, a composition of the disclosure can comprise about 500 μg chromium picolinate equivalents, derivatives, or a functional equivalent thereof.

In some embodiments, a composition of the disclosure can comprise at least about 0.001% w/w, at least about 0.0015% w/w, at least about 0.002% w/w, at least about 0.0025% w/w, at least about 0.003% w/w, at least about 0.0035% w/w, at least about 0.004% w/w, at least about 0.0045% w/w, at least about 0.005% w/w, at least about 0.0055% w/w, at least about 0.006% w/w, at least about 0.0065% w/w, at least about 0.007% w/w, at least about 0.0075% w/w, at least about 0.008% w/w, at least about 0.0085% w/w, at least about 0.009% w/w, at least about 0.0095% w/w, or at least about 0.01% w/w chromium picolinate equivalents, derivatives, or a functional equivalent thereof. In some embodiments, a composition of the disclosure can comprise at least about 0.001% w/w chromium picolinate equivalents, derivatives, or a functional equivalent thereof. In some embodiments, a composition of the disclosure can comprise at least about 0.002% w/w chromium picolinate equivalents, derivatives, or a functional equivalent thereof. In some embodiments, a composition of the disclosure can comprise at least about 0.004% w/w chromium picolinate equivalents, derivatives, or a functional equivalent thereof. In some embodiments, a composition of the disclosure can comprise at least about 0.008% w/w chromium picolinate equivalents, derivatives, or a functional equivalent thereof. In some embodiments, a composition of the disclosure can comprise between about 0.001% w/w to about 0.0025% w/w, between about 0.001% w/w to about 0.005% w/w, between about 0.001% w/w to about 0.0075% w/w, or between about 0.001% to about 0.01% w/w chromium picolinate equivalents, derivatives, or a functional equivalent thereof.

In some embodiments, magnesium malate is selected for use in embodiments of a composition of the disclosure, e.g., for its energy production, anti-inflammatory, and anti-depression effects, and as a potent inhibitor of vascular smooth muscle contraction.

In some embodiments, a composition of the disclosure can comprise about 10 mg, about 20 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,250 mg, about 1,500 mg, or about 2,000 mg cationic magnesium (Mg²⁺). In some embodiments, a composition of the disclosure can comprise about 100 mg of Mg²⁺. In some embodiments, a composition of the disclosure can comprise about 350 mg of Mg²⁺. In some embodiments, a composition of the disclosure can comprise about 700 mg of Mg²⁺. In some embodiments, a composition of the disclosure can comprise about 1000 mg of Mg²⁺.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w Mg²⁺. In some embodiments, a composition of the disclosure can comprise at least about 1.0% w/w Mg²⁺. In some embodiments, a composition of the disclosure can comprise at least about 1.3% w/w Mg²⁺. In some embodiments, a composition of the disclosure can comprise at least about 1.6% w/w Mg²⁺. In some embodiments, a composition of the disclosure can comprise at least about 2% w/w Mg²⁺. In some embodiments, a composition of the disclosure can comprise between about 0.5% w/w to 1.5% w/w, between about 1.0% w/w to about 5% w/w, between about 1.0% w/w to about 10% w/w, or between about 5% to about 10% w/w Mg²⁺.

In some embodiments, L-selenomethionine is selected for use in embodiments of a composition of the disclosure, e.g., to mitigate damage done by the degranulation of basophils and neutrophils along with microcidal molecules released by phagocytes. It increases the production of INF-gamma and IL-10, it is a component of controlling viral infections, and keeps titers of IgM and IgG normal as well as ensures adequate neutrophil chemotaxis. Selenomethionine inhibits oxidation of fats and protects vitamin E.

In some embodiments, a composition of the disclosure can comprise about 10 μg, about 20 μg, about 25 μg, about 30 μg, about 35 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 750 μg, or about 1,000 μg selenomethionine. In some embodiments, a composition of the disclosure can comprise about 250 μg selenomethionine. In some embodiments, a composition of the disclosure can comprise about 500 μg selenomethionine. In some embodiments, a composition of the disclosure can comprise about 750 μg selenomethionine. In some embodiments, a composition of the disclosure can comprise about 1000 μg selenomethionine.

In some embodiments, a composition of the disclosure can comprise at least about 0.001% w/w, at least about 0.0015% w/w, at least about 0.002% w/w, at least about 0.0025% w/w, at least about 0.003% w/w, at least about 0.0035% w/w, at least about 0.004% w/w, at least about 0.0045% w/w, at least about 0.005% w/w, at least about 0.0055% w/w, at least about 0.006% w/w, at least about 0.0065% w/w, at least about 0.007% w/w, at least about 0.0075% w/w, at least about 0.008% w/w, at least about 0.0085% w/w, at least about 0.009% w/w, at least about 0.0095% w/w, or at least about 0.01% w/w selenomethionine. In some embodiments, a composition of the disclosure can comprise at least about 0.001% w/w selenomethionine. In some embodiments, a composition of the disclosure can comprise at least about 0.002% w/w selenomethionine. In some embodiments, a composition of the disclosure can comprise at least about 0.004% w/w selenomethionine. In some embodiments, a composition of the disclosure can comprise at least about 0.008% w/w selenomethionine. In some embodiments, a composition of the disclosure can comprise between about 0.001% w/w to about 0.0025% w/w, between about 0.001% w/w to about 0.005% w/w, between about 0.001% w/w to about 0.0075% w/w, or between about 0.001% to about 0.01% w/w selenomethionine.

In some embodiments, bromelain is an enzyme selected for use in embodiments of a composition of the disclosure, e.g., to reduce swelling, inflammation, and bruising. The proinflammatory process can be modulated by compounds such as bromelain, a botanical enzyme derived from pineapple. Bromelain supplementation before and after surgery has been shown to reduce bruising, swelling, pain, and healing time. Bromelain is most commonly used to treat inflammation and soft tissue injuries, and it has been shown to speed healing from bruises and hematomas. Presurgical administration of bromelain can accelerate visible signs of healing. Bromelain has low toxicity in the recommended dosage ranges, and is generally well tolerated and free of side effects.

In some embodiments, a composition of the disclosure can comprise about 10 mg, about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg bromelain. In some embodiments, a composition of the disclosure comprises about 200 mg bromelain. In some embodiments, a composition of the disclosure comprises about 300 mg bromelain. In some embodiments, a composition of the disclosure comprises about 400 mg bromelain. In some embodiments, a composition of the disclosure comprises about 500 mg bromelain.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, or at least about 8.0% w/w bromelain. In some embodiments, a composition of the disclosure comprises at least about 0.5% w/w bromelain. In some embodiments, a composition of the disclosure comprises at least about 1.0% w/w bromelain. In some embodiments, a composition of the disclosure comprises at least about 1.5% w/w bromelain. In some embodiments, a composition of the disclosure comprises at least about 2.0% w/w bromelain.

In some embodiments, carnosine is selected for use in embodiments of a composition of the disclosure, e.g., for the benefit of immune functions including increasing the lifespan of CD 4-positive T cell clones, as well as attenuating the detrimental effects of high TNF-α concentrations.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg carnosine, its derivatives, or a functional equivalent thereof. In some embodiments, a composition of the disclosure can comprise about 1,000 mg of carnosine, its derivatives, or a functional equivalent thereof. In some embodiments, a composition of the disclosure can comprise about 2,000 mg of carnosine, its derivatives, or a functional equivalent thereof. In some embodiments, a composition of the disclosure can comprise about 2,500 mg of carnosine, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of carnosine, its derivatives, or a functional equivalent thereof. In some embodiments, a composition of the disclosure can comprise at least about 2.5% w/w carnosine, its derivatives or a functional equivalent thereof. In some embodiments, a composition of the disclosure can comprise at least about 5.0% w/w carnosine, its derivatives or a functional equivalent thereof. In some embodiments, a composition of the disclosure can comprise at least about 7.5% w/w carnosine, its derivatives or a functional equivalent thereof. In some embodiments, a composition of the disclosure can comprise at least about 9.0% w/w carnosine, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w carnosine, its derivatives, or function equivalents thereof. In some embodiments, a composition of the disclosure can comprise between about 5% w/w to about 15% w/w carnosine, its derivatives, or a functional equivalent thereof.

In some embodiments, gelatin is selected for use in embodiments of a composition of the disclosure, e.g., due to being beneficial for the overall health of the patient. Gelatin is a structural protein that is found in the human body and beneficial in helping to form joints, ligaments, skin, bones, lining of the gastrointestinal tract and hollow organs. Gelatin is made up of amino acids, including glycine, proline, hydroxyproline and arginine, all of which have benefits for digestion, wound repair, and joint support.

In some embodiments, a composition of the disclosure can comprise about 10 mg, about 20 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,250 mg, about 1,500 mg, or about 2,000 mg gelatin, gelatin derivatives, or a functional equivalent thereof. In some embodiments, a composition of the disclosure can comprise about 100 mg of gelatin, gelatin derivatives, or a functional equivalent thereof. In some embodiments, a composition of the disclosure can comprise about 350 mg of gelatin, gelatin derivatives, or a functional equivalent thereof. In some embodiments, a composition of the disclosure can comprise about 700 mg of gelatin, gelatin derivatives, or a functional equivalent thereof. In some embodiments, a composition of the disclosure can comprise about 1000 mg of gelatin, gelatin derivatives, or a functional equivalent thereof.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0% w/w, at least about 8.5% w/w, at least about 9.0% w/w, at least about 9.5% w/w, or at least about 10.0% w/w gelatin, gelatin derivatives, or a functional equivalent thereof. In some embodiments, a composition of the disclosure can comprise at least about 1.0% w/w gelatin, gelatin derivatives, or a functional equivalent thereof. In some embodiments, a composition of the disclosure can comprise at least about 1.3% w/w gelatin, gelatin derivatives, or a functional equivalent thereof. In some embodiments, a composition of the disclosure can comprise at least about 1.6% w/w gelatin, gelatin derivatives, or a functional equivalent thereof. In some embodiments, a composition of the disclosure can comprise at least about 2% w/w gelatin, gelatin derivatives, or a functional equivalent thereof. In some embodiments, a composition of the disclosure can comprise between about 0.5% w/w to 1.5% w/w, between about 1.0% w/w to about 5% w/w, between about 1.0% w/w to about 10% w/w, or between about 5% to about 10% w/w gelatin, gelatin derivatives, or a functional equivalent thereof.

In some embodiments, L-citrulline is selected for use in a composition of the disclosure, e.g., to help lower blood pressure and provide endothelial-atherogenic protection. L-citrulline is a neutral amino acid formed by enzymes in the mitochondria that also serves as a substrate for arginine. It can increase plasma and tissue levels of L-arginine and nitric oxide (NO) bioavailability. L-citrulline is a component of the urea cycle in the liver and kidneys. It increases vasodilation and counteracts insulin resistance.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg of L-citrulline or a functional equivalent thereof. In some embodiments, a composition of the disclosure can comprise about 2,000 mg of L-citrulline, its derivatives, or a functional equivalent thereof. In some embodiments, a composition of the disclosure can comprise about 3,000 mg of L-citrulline, its derivatives, or a functional equivalent thereof. In some embodiments, a composition of the disclosure can comprise about 5,000 mg of L-citrulline, its derivatives, or a functional equivalent thereof. In some embodiments, a composition of the disclosure can comprise about 7,000 mg of L-citrulline, its derivatives, or a functional equivalent thereof.

In some embodiments, a composition of the disclosure can comprise at least about 1% w/w, at least about 5% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of L-citrulline, its derivatives, or a functional equivalent thereof. In some embodiments, a composition of the disclosure can comprise at least about 5% w/w of L-citrulline, its derivatives, or a functional equivalent thereof. In some embodiments, a composition of the disclosure can comprise at least about 10% w/w of L-citrulline, its derivatives, or a functional equivalent thereof. In some embodiments, a composition of the disclosure can comprise at least about 13% w/w of L-citrulline, its derivatives, or a functional equivalent thereof. In some embodiments, a composition of the disclosure can comprise at least about 20% w/w of L-citrulline, its derivatives, or a functional equivalent thereof. In some embodiments, a composition of the disclosure can comprise at least about 25% w/w of L-citrulline, its derivatives, or a functional equivalent thereof. In some embodiments, a composition of the disclosure can comprise between about 5% w/w to about 10% w/w, between about 5% w/w to about 15% w/w, between about 5% w/w to about 20% w/w, between about 5% w/w to about 25% w/w, between about 10% w/w to about 15% w/w, between about 10% to about 20% w/w, between about 10% w/w to 25% w/w of L-citrulline, its derivatives, or a functional equivalent thereof. In some embodiments, a composition of the disclosure can comprise between about 5% w/w to about 25% w/w L-citrulline, its derivatives, or a functional equivalent thereof.

In some embodiments, arginine is selected for use in embodiments of a composition of the disclosure, e.g., to improve blood flow to the extremities and to myocutaneous flaps. NO is synthesized from arginine. Arginine is used to increase NO synthesis and bioavailability. Oral arginine supplementation is largely ineffective due to hepatic and gastrointestinal extraction of L-arginine and accompanying gastric distress. Arginine, a component of the urea cycle, is an amino acid that is intraconverted to ornithine, citrulline and agmatine. Arginine stimulates growth hormone release and insulin-like growth factor I (IGF-1), both a component of wound healing. Arginine works through different mechanisms involving both factors as well as nitric oxide, polyamine effects and increased synthesis of proline and hydroxyproline. Arginine has a positive effect in alleviating infection by increasing peritoneal macrophage phagocytic activity thereby reducing bacterial counts and increasing survival by reducing bacterial translocation from intestinal tract, restoration of weak macrophage functions, improvement in T cell function and polyamine production. This is also true when arginine is given enterally to prevent infection. Like vitamin E, arginine may be more beneficial if given prior to surgery. When given orally, arginine has a quick absorption between 21%-68% with a half-life of 1.5-2 hours. Supplementation is safe and well tolerated up to 10 g/day unless sepsis is present, then arginine is contraindicated. In some embodiments, doses of 2% of energy and given pre-operatively appear to be beneficial. The amino acid is excreted by the kidney, then almost totally reabsorbed. Given these parameters it can be given in oral doses 2-3 times a day without evidence of notable toxicity.

In some embodiments, a composition of the disclosure comprises about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, about 10,000 mg arginine. In some embodiments, a composition of the disclosure can comprise about 1,500 mg arginine. In some embodiments, a composition of the disclosure can comprise about 3,000 mg arginine. In some embodiments, a composition of the disclosure can comprise about 5,000 mg arginine. In some embodiments, a composition of the disclosure can comprise about 10,000 mg arginine.

In some embodiments, a composition of the disclosure can comprise at least about 1% w/w, at least about 5% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w arginine. In some embodiments, a composition of the disclosure can comprise at least about 25% w/w arginine. In some embodiments, a composition of the disclosure can comprise at least about 50% w/w of arginine. In some embodiments, a composition of the disclosure can comprise at least about 75% arginine. In some embodiments, a composition of the disclosure can comprise between about 5% w/w to about 15% w/w, between about 15% w/w to about 25% w/w, or between about 25% w/w to about 50% w/w arginine.

In some embodiments, branch chain amino acids (BCAAs) are selected for use in embodiments of a composition of the disclosure, e.g., to improve cachexia, albumin synthesis, immune function, and physical and mental fatigue. In some embodiments, branched-chain Keto Acids (BCKAs) are used instead of or in addition to BCAAs due to potential detrimental effects of BCAAs on ammonia production by way of enhanced catabolism of GLN in visceral tissues, the draining of alpha ketoglutarate from the TCA cycle and cataplerosis in general.

In some embodiments, a composition of the disclosure can comprise about 100 mg, about 150 mg, about 200 mg, about 210, about 220, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 500 mg, about 600 mg, about 700 mg, about 800 mg, about 900 mg, about 1000 mg, about 1100 mg, about 1125 mg, about 1150 mg, about 1175 mg, about 1200 mg, about 1300 mg, about 1400 mg, about 1500 mg, about 1600 mg, about 1700 mg, about 1800 mg, about 1900 mg, about 2000 mg branch chain amino acids and/or branched-chain keto acid equivalents, derivatives, or a functional equivalent thereof. In some embodiments, a composition of the disclosure comprises about 1125 mg L-leucine BCAA and/or BCKA equivalents, derivatives, or a functional equivalent thereof. In some embodiments, a composition of the disclosure comprises about 270 mg L-isoleucine BCAA and/or BCKA equivalents, derivatives, or a functional equivalent thereof. In some embodiments, a composition of the disclosure comprises about 270 mg L-valine BCAA and/or BCKA equivalents, derivatives, or a functional equivalent thereof. In some embodiments, a composition of the disclosure comprises about 210 mg L-Lysine HCL BCAA and/or BCKA equivalents, derivatives, or a functional equivalent thereof.

In some embodiments, a composition of the disclosure can comprise at least about 0.1% w/w, at least about 0.2% w/w, at least about 0.3% w/w, at least about 0.4% w/w, at least about 0.5% w/w, at least about 0.6% w/w, at least about 0.7% w/w, at least about 0.8% w/w, at least about 0.9% w/w, at least about 1.0% w/w, at least about 1.5% w/w, at least about 2.0% w/w, at least about 2.5% w/w, at least about 3.0% w/w, at least about 3.5% w/w, at least about 4.0% w/w, at least about 4.5% w/w, or at least about 5.0% w/w branch chain amino acids and/or branched-chain keto acid equivalents, derivatives, or a functional equivalent thereof. In some embodiments, a composition of the disclosure can comprise at least about 0.7% w/w branch chain amino acids and/or branched-chain keto acid equivalents, derivatives, or a functional equivalent thereof. In some embodiments, a composition of the disclosure can comprise at least about 0.9% w/w branch chain amino acids and/or branched-chain keto acid equivalents, derivatives, or a functional equivalent thereof. In some embodiments, a composition of the disclosure can comprise at least about 4.0% w/w branch chain amino acids and/or branched-chain keto acid equivalents, derivatives, or a functional equivalent thereof. In some embodiments, a composition of the disclosure can comprise at least about 0.7% w/w L-lysine HCL BCAA and/or BCKA equivalents, derivatives, or a functional equivalent thereof. In some embodiments, a composition of the disclosure can comprise at least about 1.0% w/w L-isoleucine BCAA and/or BCKA equivalents, derivatives, or a functional equivalent thereof. In some embodiments, a composition of the disclosure can comprise at least about 1.0% w/w L-valine BCAA and/or BCKA equivalents, derivatives, or a functional equivalent thereof. In some embodiments, a composition of the disclosure can comprise at least about 4.0% w/w L-leucine BCAA and/or BCKA equivalents, derivatives, or a functional equivalent thereof. In some embodiments, a composition of the disclosure can comprise between about 0.1% w/w to about 5.0% w/w, between 0.5% w/w to about 5.0% w/w, between about 0.7% w/w to about 5.0% w/w, between about 1.0% w/w to about 5.0% w/w, or between about 2.5% w/w to about 5.0% w/w BCAA and/or BCKA equivalents, derivatives, or a functional equivalent thereof.

In some embodiments, leucine is selected for use in embodiments of a composition of the disclosure, e.g., to serve as a substrate for protein synthesis. It exerts a stimulating effect on protein synthesis while exerting an inhibitory effect on proteolysis. It stimulates protein synthesis through the mTOR pathway, phosphorylation of ribosomal proteins and translation initiation factors. Through its role in protein anabolism, leucine has a stimulatory effect on insulin secretion and upregulation of glucose transporters. It should be noted that Leucine has been shown to reduce appetite.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2250 mg about 2,500 mg, about 2750 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg L-leucine, its derivatives, or a functional equivalent thereof. In some embodiments, a composition of the disclosure can comprise about 1,000 mg L-leucine, its derivatives, or a functional equivalent thereof. In some embodiments, a composition of the disclosure can comprise about 2,250 mg L-leucine, its derivatives, or a functional equivalent thereof. In some embodiments, a composition of the disclosure can comprise about 4,500 mg L-leucine, its derivatives, or a functional equivalent thereof. In some embodiments, a composition of the disclosure can comprise about 6,000 mg L-leucine, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w L-leucine, its derivatives, or a functional equivalent thereof. In some embodiments, a composition of the disclosure can comprise at least about 2.5% w/w L-leucine its derivatives or a functional equivalent thereof. In some embodiments, a composition of the disclosure can comprise at least about 5.0% w/w L-leucine, its derivatives or a functional equivalent thereof. In some embodiments, a composition of the disclosure can comprise at least about 7.5% w/w L-leucine, its derivatives or a functional equivalent thereof. In some embodiments, a composition of the disclosure can comprise at least about 10.0% w/w L-leucine, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w L-leucine, its derivatives, or function equivalents thereof. In some embodiments, a composition of the disclosure can comprise between about 5% w/w to about 15% w/w L-leucine, its derivatives, or a functional equivalent thereof.

In some embodiments, glutamine is selected for use in embodiments of a composition of the disclosure, e.g., due to its role in biochemical and wound healing processes by providing energy and DNA bases for rapidly proliferating cells. It accounts for approximately one third of translocated nitrogen. It protects against injury due to inflammation by inducing the expression of heat shock proteins. These proteins provide cellular protection where it becomes a modulator of injury, stress and inflammation. Glutamine given intravenously in elective surgery improves nitrogen balance, helps correct the decreased glutamine concentration found in the free intracellular skeletal muscle amino acid pool and enhances net protein synthesis most profoundly in skeletal muscle.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg glutamine, its derivatives, or a functional equivalent thereof. In some embodiments, a composition of the disclosure can comprise about 2,000 mg glutamine, its derivatives, or a functional equivalent thereof. In some embodiments, a composition of the disclosure can comprise about 3,000 mg glutamine, its derivatives, or a functional equivalent thereof. In some embodiments, a composition of the disclosure can comprise about 5,000 mg glutamine, its derivatives, or a functional equivalent thereof. In some embodiments, a composition of the disclosure can comprise about 7,000 mg glutamine, its derivatives, or a functional equivalent thereof.

In some embodiments, a composition of the disclosure can comprise at least about 1% w/w, at least about 5% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w glutamine, its derivatives, or a functional equivalent thereof. In some embodiments, a composition of the disclosure can comprise at least about 5% w/w glutamine, its derivatives, or a functional equivalent thereof. In some embodiments, a composition of the disclosure can comprise at least about 10% w/w glutamine, its derivatives, or a functional equivalent thereof. In some embodiments, a composition of the disclosure can comprise at least about 13% w/w glutamine, its derivatives, or a functional equivalent thereof. In some embodiments, a composition of the disclosure can comprise at least about 20% w/w glutamine, its derivatives, or a functional equivalent thereof. In some embodiments, a composition of the disclosure can comprise at least about 25% w/w glutamine, its derivatives, or a functional equivalent thereof. In some embodiments, a composition of the disclosure can comprise between about 5% w/w to about 10% w/w, between about 5% w/w to about 15% w/w, between about 5% w/w to about 20% w/w, between about 5% w/w to about 25% w/w, between about 10% w/w to about 15% w/w, between about 10% to about 20% w/w, between about 10% w/w to 25% w/w glutamine, its derivatives, or a functional equivalent thereof. In some embodiments, a composition of the disclosure can comprise between about 5% w/w to about 25% w/w glutamine, its derivatives, or a functional equivalent thereof.

In some embodiments, ashwagandha is selected for use in embodiments of a composition of the disclosure, e.g., due to its wide ranging health benefits. It is a GABA mimetic and has been shown to promote formation of dendrites. It exerts an anxiolytic effect and improves energy levels and mitochondrial health. Ashwagandha strengthens the body's resilience to stress. It improves the body's defense against disease by improving the cell-mediated immunity. Ashwagandha has potent antioxidant properties that protect against free radical cellular damage.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2250 mg about 2,500 mg, about 2750 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg ashwagandha, its derivatives, or a functional equivalent thereof. In some embodiments, a composition of the disclosure can comprise about 750 mg ashwagandha, its derivatives, or a functional equivalent thereof. In some embodiments, a composition of the disclosure can comprise about 1,500 mg ashwagandha, its derivatives, or a functional equivalent thereof. In some embodiments, a composition of the disclosure can comprise about 3,000 mg ashwagandha, its derivatives, or a functional equivalent thereof. In some embodiments, a composition of the disclosure can comprise about 5,000 mg ashwagandha, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w ashwagandha, its derivatives, or a functional equivalent thereof. In some embodiments, a composition of the disclosure can comprise at least about 2.5% w/w ashwagandha its derivatives or a functional equivalent thereof. In some embodiments, a composition of the disclosure can comprise at least about 5.0% w/w ashwagandha, its derivatives or a functional equivalent thereof. In some embodiments, a composition of the disclosure can comprise at least about 7.5% w/w ashwagandha, its derivatives or a functional equivalent thereof. In some embodiments, a composition of the disclosure can comprise at least about 10.0% w/w ashwagandha, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w ashwagandha, its derivatives, or function equivalents thereof. In some embodiments, a composition of the disclosure can comprise between about 5% w/w to about 15% w/w ashwagandha, its derivatives, or a functional equivalent thereof.

In some embodiments, Holy Basil is selected for use in embodiments of a composition of the disclosure, e.g., to aid in protection of tissues against physical, chemical, and metabolic stresses. It helps balance blood glucose, blood pressure, and lipid levels. It exerts positive effects on memory and cognitive function and protects against psychological stress with its anxiolytic and antidepressant properties. It contains ocimumosides and cerebrosides which display anti-stress effects by normalizing plasma corticosterone, hyperglycemia, plasma creatine kinase, and adrenal hypertrophy. Ocimum sanctum can lower elevated cortisol levels and potentially regulating corticosteroid-induced diabetes mellitus.

In some embodiments, a composition of the disclosure can comprise about 100 mg, about 200 mg, about 300 mg, about 400 mg, about 500 mg, about 600 mg, about 700 mg, about 800 mg, about 900 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, about 9,500 mg, or about 10,000 mg Ocimum sanctum equivalents, its derivatives, or a functional equivalent thereof. In some embodiments, a composition of the disclosure can comprise about 1,000 mg Ocimum sanctum equivalents, its derivatives, or a functional equivalent thereof. In some embodiments, a composition of the disclosure can comprise about 2,000 mg Ocimum sanctum equivalents, its derivatives, or a functional equivalent thereof. In some embodiments, a composition of the disclosure can comprise about 4,000 mg Ocimum sanctum equivalents, its derivatives, or a functional equivalent thereof. In some embodiments, a composition of the disclosure can comprise about 6,000 mg Ocimum sanctum equivalents, its derivatives, or a functional equivalent thereof.

In some embodiments, a composition of the disclosure can comprise at least about 1% w/w, at least about 5% w/w, at least about 7.5%, at least about 9.0% w/w, at least about 10% w/w, at least about 12.5% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w Ocimum sanctum equivalents, its derivatives, or a functional equivalent thereof. In some embodiments, a composition of the disclosure can comprise at least about 2.5% w/w Ocimum sanctum equivalents its derivatives or a functional equivalent thereof. In some embodiments, a composition of the disclosure can comprise at least about 5.0% w/w Ocimum sanctum equivalents, its derivatives or a functional equivalent thereof. In some embodiments, a composition of the disclosure can comprise at least about 7.5% w/w Ocimum sanctum equivalents, its derivatives or a functional equivalent thereof. In some embodiments, a composition of the disclosure can comprise at least about 9.0% w/w Ocimum sanctum equivalents, its derivatives, or a functional equivalent thereof. In some embodiments, a composition of the disclosure can comprise between about 1% w/w and about 5% w/w, between about 5% w/w to about 10% w/w, between about 5% to about 15% w/w, between about 5% to about 20% w/w, between about 10% w/w to about 15% w/w, or between about 10% w/w to about 20% w/w Ocimum sanctum equivalents, its derivatives, or function equivalents thereof. In some embodiments, a composition of the disclosure can comprise between about 5% w/w to about 15% w/w Ocimum sanctum equivalents, its derivatives, or a functional equivalent thereof.

In some embodiments, Arnica montana is selected for use in embodiments of a composition of the disclosure, e.g., to aid in postoperative pain, postoperative ecchymosis, and edema, as well as postoperative drainage. Arnica montana is the Latin name for a perennial with bright, yellow daisy-like flowers Arnica can be used as an ointment, gel, or cream on unbroken skin or taken internally when it is diluted homeopathically. It can also support postoperative hemoglobin levels.

In some embodiments, a composition of the disclosure can comprise about 1 pellet, about 2 pellets, about 3 pellets, about 4 pellets, about 5 pellets, about 6 pellets, about 7 pellets, about 8 pellets, about 9 pellets, or about 10 pellets of Arnica montana 200c equivalents and its derivatives, or a functional equivalent thereof. In some embodiments, a composition of the disclosure can comprise about 2 pellets of Arnica montana 200c equivalents and its derivatives, or a functional equivalent thereof. In some embodiments, a composition of the disclosure can comprise about 5 pellets of Arnica montana 200c equivalents and its derivatives, or a functional equivalent thereof. In some embodiments, a composition of the disclosure can comprise about 7 pellets of Arnica montana 200c equivalents and its derivatives, or a functional equivalent thereof.

In some embodiments, ingredients of a composition as disclosed herein can be in a pharmaceutically-acceptable, physiologically-acceptable, or nutritionally-acceptable form suitable for human consumption. In some embodiments, amino acid ingredients of a composition as disclosed herein can be in a free form, salt, prodrug, homodimer, heterodimer from thereof. In some embodiments, ingredients of a composition as disclosed herein can be in a form that adheres to established guidelines for human consumption. In some embodiments, ingredients of a composition as disclosed herein can be in a form suitable for human consumption. In some embodiments, ingredients of a composition as disclosed herein can be in salt form. In some embodiments, ingredients of a composition as disclosed herein can be in an ionic form. In some embodiments, ingredients of a composition as disclosed herein can be in a prodrug form.

### Nutrient Modulation

In some embodiments, compositions and methods of the disclosure comprise administering a nutrient modulating composition. In some embodiments, a nutrient modulating composition comprises a nutritional supplement.

As described herein, a nutrient modulating composition can modulate a nutrient level in a subject. In some embodiments, modulating a nutrient level in a subject can comprise increasing or decreasing a nutrient level in a subject. In some embodiments, a nutrient level in a subject or a cell of a subject can be increased or decreased by modulating nutrient levels in a composition administered to the subject. In some embodiments, a nutrient level in a subject or a cell of a subject can be increased or decreased by modulating nutrient levels in a nutritional supplement administered to the subject.

In some embodiments, the nutrient modulated is a vitamin. In some embodiments, the nutrient modulated is a mineral. In some embodiments, the nutrient modulated is a carbohydrate. In some embodiments, the nutrient modulated is a saccharide. In some embodiments, the nutrient modulated is a protein. In some embodiments, the nutrient modulated is an enzyme. In some embodiments, the nutrient modulated is a peptide. In some embodiments, the nutrient modulated is a lipid. In some embodiments, the nutrient is a fatty acid.

In some embodiments, a nutritional supplement changes the level of a metabolite or nutrient in a cell. In some embodiments, a nutritional supplement increases the level of a metabolite or a nutrient in a cell. In some embodiments, a nutritional supplement decreases the level of a metabolite or a nutrient in a cell. In some embodiments, a nutritional supplement changes the level of a metabolite or nutrient in systemic circulation or healthy tissues. In some embodiments, a nutritional supplement changes the average level of a metabolite or nutrient in plasma compared to a baseline level. In some embodiments, a nutritional supplement changes the level of a metabolite or nutrient associated with the microbiota (e.g., gut microbiota).

In some embodiments, a nutrient level in a subject or a cell of a subject can be increased. In some embodiments, a nutrient level in a subject or a cell of a subject can be increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 500%, at least about 750%, at least about 1000%, or at least about 10,000%.

In some embodiments, a nutrient level in a subject can be increased in a subject or a cell of a subject by providing the nutrient in a composition in an amount greater than the daily recommended nutrient level for the subject. In some embodiments, a nutrient level in a subject or a cell of a subject can be increased in a subject by providing the nutrient in a composition in an amount that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 100%, at least about 500%, at least about 1000%, at least about 5000%, or at least about 10000% greater than the daily recommended nutrient level for the subject.

In some embodiments, an average nutrient level in plasma can be increased compared to a baseline level of nutrient in the subject. In some embodiments, an average nutrient level in plasma can be increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 100%, at least about 500%, at least about 1000%, at least about 5000%, or at least about 10000% compared to a baseline level of the nutrient in the subject.

In some embodiments, an average nutrient level in plasma can be increased in a subject by providing the subject a composition comprising the nutrient in an amount greater than the daily recommended nutrient level for the subject. In some embodiments, an average nutrient level in plasma can be increased in a subject by providing a composition comprising the nutrient in an amount that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 100%, at least about 500%, at least about 1000%, at least about 5000%, or at least about 10000% greater than the daily recommended nutrient level for the subject.

In some embodiments, a nutritional supplement provides more than a RDA of a nutrient. In some embodiments, a nutritional supplement provides greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of a RDA of a nutrient.

In some embodiments, a nutritional supplement comprises more than a RDA of a nutrient. In some embodiments, a nutritional supplement comprises greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of a RDA of a nutrient.

In some embodiments, a dietary product provides more than an average recommended dietary intake of a nutrient. In some embodiments, a dietary product provides greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of an average recommended dietary intake of a nutrient.

In some embodiments, a dietary product comprises more than an average recommended dietary intake of a nutrient. In some embodiments, a dietary product provides greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of an average recommended dietary intake of a nutrient.

In some embodiments, a dietary product provides more than a daily median intake of a nutrient. In some embodiments, a dietary product provides greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of a daily median intake of a nutrient.

In some embodiments, a dietary product comprises more than a daily median intake of a nutrient. In some embodiments, a dietary product provides greater than about 1%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 60%, greater than about 65%, greater than about 65%, greater than about 70%, greater than about 75%, greater than about 80%, greater than about 85%, greater than about 90%, greater than about 100%, greater than about 500% greater than about 1000%, greater than about 5000%, or greater than about 10000% of a daily median intake of a nutrient.

In some embodiments, a nutrient level in a subject or a cell of a subject can be decreased. In some embodiments, a nutrient level in a subject or a cell of a subject can be decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In some embodiments, a nutrient level in a subject can be decreased in a subject or a cell of a subject by providing the nutrient in a composition in an amount less than the daily recommended nutrient level for the subject. In some embodiments, a nutrient level in a subject or a cell of a subject can be decreased in a subject by providing the nutrient in a composition in an amount that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% less than the daily recommended nutrient level for the subject.

In some embodiments, an average nutrient level in plasma can be decreased compared to a baseline level of the nutrient. In some embodiments, an average nutrient level in plasma can be decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% compared to a baseline level of the nutrient.

In some embodiments, an average nutrient level in plasma can be decreased in a subject by providing the subject a composition comprising the nutrient in an amount less than the daily recommended nutrient level for the subject. In some embodiments, an average nutrient level in plasma can be decreased in a subject by providing a composition comprising the nutrient in an amount that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% less than the daily recommended nutrient level for the subject.

In some embodiments, a nutritional supplement provides less than a RDA of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of a RDA of a nutrient.

In some embodiments, a nutritional supplement comprises less than a RDA of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of a RDA of a nutrient.

In some embodiments, a dietary product provides less than an average recommended dietary intake of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of an average recommended dietary intake of a nutrient.

In some embodiments, a dietary product comprises less than an average recommended dietary intake of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of an average recommended dietary intake of a nutrient.

In some embodiments, a dietary product provides less than a daily median intake of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of a daily median intake of a nutrient.

In some embodiments, a dietary product comprises less than a daily median intake of a nutrient. In some embodiments, a dietary product provides less than 99.9%, less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1%, less than 0.5%, less than 0.1%, less than 0.01%, or less than 0.001% of a daily median intake of a nutrient.

### Pharmaceutical and Nutritional Compositions

A nutritional composition of the disclosure can be used, for example, before, during, or after treatment of a subject with, for example, a pharmaceutical agent or therapeutic modality (e.g., surgery).

Subjects can be, for example, elderly adults, adults, adolescents, pre-adolescents, children, toddlers, infants, neonates, and non-human animals. In some embodiments, a subject is a patient.

A composition of the disclosure can be a combination of any compositions or nutritional products described herein with other chemical components, such as carriers, stabilizers, diluents, dispersing agents, suspending agents, thickening agents, flavorants, sweeteners, flavor masking agents, processing agents, and/or excipients. The pharmaceutical composition facilitates administration of the dietary product to a subject. Pharmaceutical or nutritional compositions can be administered in nutritionally-effective amounts as pharmaceutical or nutritional compositions by various forms and routes including, for example, intravenous, subcutaneous, intramuscular, oral, parenteral, ophthalmic, subcutaneous, transdermal, nasal, vaginal, topical, via tube, or via nasogastric tube. In some embodiments, the composition or dietary product of the disclosure is administered orally.

For oral administration, nutritional compositions can be formulated by combining the active compositions or nutritional products of the disclosure with pharmaceutically-acceptable or nutritionally-acceptable carriers, excipients, diluents, or solvents. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Nutritional compositions can be formulated for intravenous administration. The nutritional compositions can be in a form suitable for parenteral injection as a sterile suspension, solution or emulsion in oily or aqueous vehicles, and can contain formulatory agents such as suspending, stabilizing and/or dispersing agents. Nutritional compositions for parenteral administration include aqueous solutions of the active dietary products in water-soluble form. Suspensions of the active dietary products can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the dietary products to allow for the preparation of highly concentrated solutions. Alternatively, the active ingredient can be in powder form for constitution with a suitable vehicle, e.g., sterile pyrogen-free water, before use.

Nutritional compositions can be formulated using one or more physiologically-acceptable carriers comprising excipients and auxiliaries that facilitate processing of the active dietary products into preparations that can be used pharmaceutically or nutritionally. Formulations can be modified depending upon the route of administration chosen. Compositions comprising a nutritional product described herein can be manufactured, for example, by mixing, dissolving, emulsifying, encapsulating, entrapping, or compression processes.

The pharmaceutical compositions can include at least one physiologically-acceptable, pharmaceutically-acceptable, or nutritionally-acceptable carrier, diluent, or excipient and dietary products described herein as free-base or pharmaceutically-acceptable salt form. Compositions can contain solubilizers, stabilizers, tonicity enhancing agents, buffers and preservatives.

Methods for the preparation of compositions comprising the dietary products described herein include formulating the nutritional products with one or more inert, physiologically-acceptable, pharmaceutically-acceptable, or nutritionally-acceptable excipients, diluents, carriers, or solvents to form a solid, semi-solid, or liquid composition. Solid compositions include, for example, powders, tablets, dispersible granules, capsules, and cachets. Liquid compositions include, for example, solutions in which a dietary product is dissolved, emulsions comprising a dietary product, or a solution containing liposomes, micelles, or nanoparticles comprising a dietary product as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other physiologically-acceptable, pharmaceutically-acceptable, or nutritionally-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of physiologically-acceptable, pharmaceutically-acceptable, or nutritionally-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

Anti-adherent: A composition of the disclosure can comprise an anti-adherent. In some embodiments, a composition of the disclosure can comprise an anti-adherent, such as magnesium stearate.

Binding agent: A composition of the disclosure can comprise at least one binding agent to hold the composition together. In some embodiments, a composition of the disclosure can comprise a binding agent, such as a saccharide, protein, or synthetic polymer. In some embodiments, a composition of the disclosure can comprise a disaccharide (e.g., sucrose or lactose), a polysaccharide or polysaccharide derivative (e.g., starch, cellulose, modified cellulose, cellulose ether), or a sugar alcohol (e.g., xylitol, sorbitol, or mannitol). In some embodiments, a composition of the disclosure can comprise a protein binder, such as gelatin. In some embodiments, a composition of the disclosure can comprise a synthetic polymer binder, such as polyvinylpyrrolidone (PVP) or polyethylene glycol (PEG).

Preservative: A composition of the disclosure can comprise at least one preservative. In some embodiments, a composition of the disclosure can comprise an antioxidant or an antimicrobial. Antioxidant agents delay or prevent the deterioration of the composition by oxidative mechanisms. Antimicrobial agents inhibit the growth of spoilage or pathogenic microorganisms in the composition.

Antioxidant: In some embodiments, an antioxidant agent is added to the composition to delay or prevent autooxidation of unsaturated fatty acids or enzyme-catalyzed oxidation. In some embodiments, a composition of the disclosure comprises at least one of ascorbic acid, butylated hydroxyanisole (BHA), butylated hydroxytoluene (BHT), citric acid, a sulfite, tertiary butylhydroquinone (TBHQ), or a tocopherol. In some embodiments, a composition of the disclosure comprises ascorbic acid. In some embodiments, a composition of the disclosure comprises BHT. In some embodiments, a composition of the disclosure comprises citric acid.

In some embodiments, a composition of the disclosure comprises about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg of an antioxidant agent. In some embodiments, a composition of the disclosure comprises up to about 100 mg/kg, up to about 200 mg/kg, up to about 300 mg/kg, up to about 400 mg/kg, up to about 500 mg/kg, up to about 600 mg/kg, up to about 700 mg/kg, up to about 800 mg/kg, up to about 900 mg/kg, or up to about 1000 mg/kg of an antioxidant agent.

Antimicrobial agent: In some embodiments, an antimicrobial agent is added to the composition to delay or prevent growth of spoilage or pathogenic microorganisms in the composition. In some embodiments, a composition of the disclosure comprises at least one of acetic acid, benzoic acid, natamycin, nisin, a nitrate, a nitrite, propionic acid, sorbic acid, a sulfite, or sulfur dioxide.

In some embodiments, a composition of the disclosure comprises about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg of an antimicrobial agent. In some embodiments, a composition of the disclosure comprises up to about 100 mg/kg, up to about 200 mg/kg, up to about 300 mg/kg, up to about 400 mg/kg, up to about 500 mg/kg, up to about 600 mg/kg, up to about 700 mg/kg, up to about 800 mg/kg, up to about 900 mg/kg, or up to about 1000 mg/kg of an antimicrobial agent.

Colorants: A composition of the disclosure can comprise at least one colorant. In some embodiments, a composition of the disclosure comprises a natural colorant or a synthetic colorant. In some embodiments, a composition of the disclosure comprises a natural colorant. In some embodiments, a composition of the disclosure comprises an anthocyanin. In some embodiments, a composition of the disclosure comprises an anthocyanin, such as pelargonidin-3-glucoside obtained from strawberries (Fragaria species) or malvidin-3-glucoside obtained from grapes (Vitis species). In some embodiments, a composition of the disclosure comprises a betacyanin. In some embodiments, a composition of the disclosure comprises a betacyanin, such as betanin obtained from beet root (Beta vulgaris). In some embodiments, a composition of the disclosure comprises a carotenoid. In some embodiments, a composition of the disclosure comprises a carotenoid, such as bixin obtained from annatto (Bixa Orellana); crocin obtained from saffron (Crocus sativus); capsanthin obtained from paprika (Capsicum annuum); beta-carotene obtained from carrot (Daucus carota subsp. sativus); or canthaxanthin obtained from mushrooms (Cantharellus cinnabarinus). In some embodiments, a composition of the disclosure comprises a phenolic. In some embodiments, a composition of the disclosure comprises a phenolic, such as curcumin obtained from turmeric (Curcuma longa).

In some embodiments, a composition of the disclosure comprises a synthetic colorant. In some embodiments, a composition of the disclosure comprises allura red AC, brilliant blue FCF, erythrosine, fast green FCF, indico carmine, sunset yellow FCF, or tartrazine. In some embodiments, a composition of the disclosure comprises FD&C red no. 40, FD&C blue no. 1, FD&C red no. 3, FD&C green no. 3, FD&C blue no. 2, FD&C yellow no. 6, or FD&C yellow no. 5. In some embodiments, a composition of the disclosure comprises E133, E127, E132, E110, or E102.

Flavorants: A composition of the disclosure can comprise at least one flavoring agent. In some embodiments, a composition of the disclosure can comprise a natural flavoring substance, a nature-identical flavoring substance, or an artificial flavoring substance. In some embodiments, a composition of the disclosure can comprise a natural flavoring substance, such as a spice, fruit juice, or vegetable juice. In some embodiments, a composition of the disclosure can comprise natural berry flavor. In some embodiments, a composition of the disclosure can comprise a nature-identical flavoring substance, such as vanillin.

In some embodiments, a composition of the disclosure can comprise an artificial flavoring substance, such as allylpyrazine, methoxypyrazine, 2-iso-butyl-3-methoxypyrazine, acetyl-L-pyrazine, 2-acetoxy pyrazine, aldehydes, alcohols, esters, ketones, pyrazines, phenolics, or terpenoids.

Sweetener: A composition of the disclosure can comprise at least one sweetener. In some embodiments, a composition of the disclosure comprises sucrose, glucose, fructose, corn syrup, high-fructose corn syrup, a natural extract, or a sugar alcohol. In some embodiments, a composition of the disclosure comprises a natural extract, such as Luo Han Guo (monkfruit) extract or Stevia extract.

Luo Han Guo (Monk Fruit) Extract taken from the fruit of an herbaceous perennial vine native to Asia (Siraitia grosvenorii) is selected for use in a composition of the disclosure, e.g., due to its natural anti-inflammatory properties. It is full of nutrients and is rich in antioxidants. Monk fruit does not impact blood sugar levels. Its sweetness comes from a compound called mogroside, a compound that may help maintain blood glucose metabolism. It does this through up-regulating the expression of phosphatidylinositol-3-kinase (PI3K), glucose transporter type 2 (GLUT2), and glycogen synthesis (GS). It also down-regulates phosphorylated insulin receptor substrate-1 (p-IRS-1(ser)) and glycogen synthesis kinase-3β (p-GSK-3β). Insulin resistance and increased glycogen synthesis were increased through the PI3K/Akt pathway.

Flavor masking agents: A composition of the disclosure can comprise at least one flavor masking agent. Non-limiting examples of a flavor masking agent include a bitter blocking agent, a protein masking agent, an acid masking agent, a preservative masking agent, a vitamin masking agent, a cyclodextrin, a microencapsulation agent, a sweetness enhancer, a particle coating agent.

Processing agents: A composition of the disclosure can comprise at least one processing additive. In some embodiments, a composition of the disclosure can comprise an anticaking agent, a bleaching agent, a chelating agent, a clarifying agent, conditioning agent, emulsifying agent, a humectant, a pH control agent, a stabilizing agent, or a thickening agent. In some embodiments, a composition of the disclosure can comprise an anticaking agent such as sodium aluminosilicate, a chelating agent such as ethylenediaminetetraacetic acid (EDTA), a conditioning agent such as potassium bromate, or a pH control agent such as citric acid or lactic acid. In some embodiments, a composition of the disclosure can comprise a chelating agent such as sodium gluconate. In some embodiments, a composition of the disclosure can comprise a humectant such as glycerol, or a stabilizing and thickening agent.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Nutritional compositions described herein can be in unit dosage forms suitable for single administration of precise dosages. In unit dosage form, the formulation is divided into unit doses containing appropriate quantities of one or more nutritional products. The unit dosage can be in the form of a package containing discrete quantities of the formulation. Non-limiting examples are packaged injectables, vials, sachets, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

A composition of the disclosure can comprise at least one pharmaceutical excipient, such as an anti-adherent, a binder, coating, colorant, disintegrant, flavorant, flavor masking agent, preservative, sorbent, sweetener, or vehicle. In some embodiments, a composition of the disclosure comprises a colorant and a flavorant. In some embodiments, a composition of the disclosure comprises a colorant, flavorant, and sweetener. In some embodiments, a composition of the disclosure comprises a flavorant, sweetener, and a preservative. In some embodiments, a composition of the disclosure comprises a flavorant, sweetener, preservative, and flavor masking agent.

In some embodiments, a composition of the disclosure comprises trehalose, arabinogalactan, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, and retinyl palmitate, or a functional equivalent thereof. In some embodiments, a composition of the disclosures include L-citrulline, arginine, sodium citrate, potassium aspartate, bromelain, copper, zinc, carnosine, manganese, retinoic acid, branch chain amino acids, leucine, chromium, glutamine, gelatin, ashwagandha (Withania somnifera), Holy Basil, Arnica montana, citric acid, or combinations thereof. In some embodiments, a composition of the disclosure can comprise one or more flavorings, sweeteners, or excipients, or diluents. In some embodiments, the one or more flavorings comprise monk fruit, natural berry flavor, other natural or artificial flavors, or combinations thereof.

### Dosing

Nutritional compositions described herein can be in unit dosage forms suitable for single administration of precise dosages. In unit dosage form, the formulation is divided into unit doses containing appropriate quantities of one or more dietary products. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A dose can be expressed in terms of an amount of a nutrient divided by the mass of the subject, for example, milligrams per kilograms of subject body mass. A dose can be expressed in terms of an amount of nutrient divided by the total mass of the nutritional composition, for example, milligrams per gram of nutritional composition. A dose can be expressed as a percentage of the weight of the nutrient over the total weight of the composition (% w/w).

A composition described herein can be administered immediately before or immediately after a meal. A composition described here can be administered within about 5 minutes, about 10 minutes, about 15 minutes, about 20 minutes, about 25 minutes, about 30 minutes, about 40 minutes, about one hour, about 2 hours, about 3 hours, about 4 hours, about 5 hours, or about 6 hours before or after a meal. In some embodiments, a composition described herein can be administered with water.

A composition described herein can be in unit dosage forms suitable for single administration of precise dosages. In unit dosage form, the formulation is divided into unit doses containing appropriate quantities of the composition. In some embodiments, the unit dosage can be in the form of a package containing discrete quantities of the formulation. In some embodiments, formulations of the disclosure can be presented in unit dosage form in single-serving sachet. In some embodiments, formulations of the disclosure can be presented in a single-dose non-reclosable container. In some embodiments, a formulation of the disclosure can be presented in a reclosable container, and the subject can obtain a single-dose serving of the formulation using a scoop or spoon designed to distribute a single-dose serving. In some embodiments, a formulation of the disclosure can be presented in a reclosable container, and the subject can obtain a single-dose serving of the formulation using a scoop or spoon designed to distribute a half-dose serving (i.e., two scoops to distribute one serving).

In some embodiments, a nutritionally-effective dose of a composition can be packaged as a sachet suitable for human consumption. In some embodiments, a nutritionally-effective dose of a composition can be packaged as a sachet suitable for human consumption, wherein the composition is dissolvable in a solvent. In some embodiments, a nutritionally-effective dose of a composition can be packaged as a sachet suitable for human consumption, wherein the ingredients of the composition are mixed into a solvent to provide a potable mixture that is suitable for human consumption.

A composition described herein can be present in a unit dose serving in a range from about 1 g to about 2 g, from about 2 g to about 3 g, from about 3 g to about 4 g, from about 4 g to about 5 g, from about 5 g to about 6 g, from about 6 g to about 7 g, from about 7 g to about 8 g, from about 8 g to about 9 g, from about 9 g to about 10 g, from about 10 g to about 11 g, from about 11 g to about 12 g, from about 12 g to about 13 g, from about 13 g to about 14 g, from about 14 g to about 15 g, from about 15 g to about 16 g, from about 16 g to about 17 g, from about 17 g to about 18 g, from about 18 g to about 19 g, from about 19 g to about 20 g, from about 20 g to about 21 g, from about 21 g to about 22 g, from about 22 g to about 23 g, from about 23 g to about 24 g, or from about 24 g to about 25 g.

A composition described herein can be present in a unit dose serving in an amount of about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, or about 25 g. In some embodiments, a composition described herein is present in a unit dose serving in an amount of about 10 g, about 13 g, about 16 g, about 20 g, or about 24 g.

In some embodiments, a composition described herein is present in a unit dose serving in an amount of about 13 g. In some embodiments, a composition described herein is present in a unit dose serving in a sachet in an amount of about 13 g. In some embodiments, a composition described herein is present in a unit dose serving in an amount of about 16 g. In some embodiments, a composition described herein is present in a unit dose serving in a sachet in an amount of about 16 g. In some embodiments, a composition described herein is present in a unit dose serving in an amount of about 20 g. In some embodiments, a composition described herein is present in a unit dose serving in a sachet in an amount of about 20 g.

A composition described herein can be provided to a subject in one or more servings per day. In some embodiments, 1 serving, 2 servings, 3 servings, 4 servings, 5 servings, 6 servings, 7 servings, 8 servings, 9 servings, 10 servings, 11 servings, or 12 servings of a composition described herein is provided to a subject in one day. In some embodiments, 2 servings of a composition described herein is provided to a subject in one day. In some embodiments, 3 servings of a composition described herein is provided to a subject in one day.

A composition disclosed herein can be administered as soon as is practical prior to the onset of a therapeutic treatment regimen, (e.g., surgery). In some embodiments, the length of time a composition can be administered is about 12 hours, about 24 hours, about 36 hours, or about 48 hours. In some embodiments, the length of time a composition can be administered is about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days. In some embodiments, the length of time a composition can be administered is about is about 1 week, about 2 weeks, about 3 weeks, or about 4 weeks.

In some embodiments, a therapeutically effective amount of a composition of the disclosure can be administered to a subject. In some embodiments, a therapeutically effective amount of a composition of the disclosure is from about 0.1 g/kg/day to about 0.2 g/kg/day, from about 0.2 g/kg/day to about 0.3 g/kg/day, from about 0.3 g/kg/day to about 0.4 g/kg/day, from about 0.4 g/kg/day to about 0.5 g/kg/day, from about 0.1 g/kg/day to about 0.5 g/kg/day, from about 0.5 g/kg/day to about 0.6 g/kg/day, from about 0.6 g/kg/day to about 0.7 g/kg/day, from about 0.7 g/kg/day to about 0.8 g/kg/day, from about 0.8 g/kg/day to about 0.9 g/kg/day, from about 0.9 g/kg/day to about 1.0 g/kg/day, from about 0.5 g/kg/day to about 1.0 g/kg/day, from about 1.0 g/kg/day to about 1.1 g/kg/day, from about 1.1 g/kg/day to about 1.2 g/kg/day, from about 1.2 g/kg/day to about 1.3 g/kg/day, from about 1.3 g/kg/day to about 1.4 g/kg/day, from about 1.4 g/kg/day to about 1.5 g/kg/day, or from about 1.0 g/kg/day to about 1.5 g/kg/day. In some embodiments, a therapeutically effective amount of a composition of the disclosure is from 0.1 g/kg/day to about 0.5 g/kg/day.

### Methods of Use

In some embodiments, disclosed herein is a method comprising contacting a composition with a diluent or excipient to provide a mixture. In some embodiments, disclosed herein is a method comprising contacting a composition with a diluent to provide a mixture. In some embodiments, disclosed herein is a method comprising contacting a composition with a excipient to provide a mixture. In some embodiments, the method further comprises mixing the mixture to obtain in the mixture a desired consistency. In some embodiments, the method further comprises mixing the mixture to obtain a consistency that is suitable for human consumption. In some embodiments, the method further comprises mixing the composition into a solvent to provide a potable mixture that is suitable for human consumption. In some embodiments, the method further comprises mixing the composition into water to provide a potable mixture that is suitable for human consumption.

In some embodiments, a composition as disclosed herein can be administered to a subject prior to performing surgery on the subject. In some embodiments, a composition as disclosed herein can be administered to a subject in a plurality of doses prior to performing surgery on the subject. In some embodiments, a composition as disclosed herein can be administered to a subject in at least 1 dose, at least 2 doses, at least 3 doses, at least 4 doses, at least 5 doses, at least 6 doses, at least 7 doses, at least 8 doses, at least 9 doses, or at least 10 doses prior to performing surgery on the subject. In some embodiments, a composition as disclosed herein can be administered to a subject in at least 2 doses prior to performing surgery on the subject.

The present disclosure provides methods for administering a composition to a subject. A composition disclosed herein can be administered to a subject prior to the subject undergoing a surgical procedure. In some embodiments, a nutritionally-effective amount of a composition can be administered to a subject prior to the subject undergoing a surgical procedure. In some embodiments, a nutritionally-effective amount of a composition can be administered in a plurality of doses prior to a subject prior to the subject undergoing a surgical procedure. In some embodiments, a nutritionally-effective amount of a composition can be administered in a plurality of separate doses prior to a subject prior to the subject undergoing a surgical procedure. In some embodiments, a nutritionally-effective amount of a composition can be administered in a plurality of doses between about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, or about 48 hours prior to undergoing surgery. In some embodiments, a nutritionally-effective amount of a composition can be administered in a plurality of doses between about 2 hours and about 6 hours, between about 2 hours and about 12 hours, between about 2 hours and about 18 hours, between about 2 hours and about 24 hours, between about 2 hours and about 36 hours, or between about 2 hours and 48 hours prior to undergoing surgery.

In some embodiments, a first nutritionally-effective dose of a composition can be administered to a subject and after a period of time, a second nutritionally-effective dose of a composition is administered to a subject. In some embodiments, a nutritionally-effective dose of a composition can be administered to a subject and after a period of about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, or about 48 hours before a second nutritionally-effective dose of a composition is administered to a subject. In some embodiments, a first nutritionally effective dose of a composition can be administered can be administered to a subject and after a period of about 8 hours a second nutritionally-effective dose of a composition is administered to a subject. In some embodiments, a first nutritionally effective dose of a composition can be administered to a subject and after a period of about 10 hours a second nutritionally-effective dose of a composition is administered to a subject. In some embodiments, a first nutritionally-effective dose of a composition can be administered can be administered to a subject and after a period of about 12 hours a second nutritionally-effective dose of a composition is administered to a subject.

In some embodiments, a first nutritionally-effective dose of a composition can be administered to a subject about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, or about 48 hours prior to surgery and a second nutritionally-effective dose of a composition administered to a subject about 1 hour, about 2 hours, about 3 hours, about 4 hours, about 5 hours, about 6 hours, about 7 hours, about 8 hours, about 9 hours, about 10 hours, about 11 hours, or about 12 hours prior to surgery. In some embodiments, a first nutritionally-effective dose of a composition can be administered to a subject about 12 hours prior to surgery and a second nutritionally-effective dose of a composition administered to a subject about 2 hours prior to surgery. In some embodiments, a first nutritionally-effective dose of a composition can be administered to a subject about 12 hours prior to surgery and a second nutritionally-effective dose of a composition administered to a subject about 4 hours prior to surgery. In some embodiments, a first nutritionally-effective dose of a composition can be administered to a subject about 12 hours prior to surgery and a second nutritionally-effective dose of a composition administered to a subject about 6 hours prior to surgery. In some embodiments, a first nutritionally-effective dose of a composition can be administered to a subject about 6 hours prior to surgery and a second nutritionally-effective dose of a composition administered to a subject about 2 hours prior to surgery. In some embodiments, a first nutritionally-effective dose of a composition can be administered to a subject about 8 hours prior to surgery and a second nutritionally-effective dose of a composition administered to a subject about 2 hours prior to surgery. In some embodiments, a first nutritionally-effective dose of a composition can be administered to a subject about 12 hours prior to surgery and a second nutritionally-effective dose of a composition administered to a subject about 2 hours prior to surgery.

In some embodiments, a nutritionally-effective dose of a composition is not administered to a subject within about 1 hour, about 2 hours, about 3 hours, about 4 hours, about 5 hours, or about 6 hours prior to surgery. In some embodiments, a nutritionally-effective dose of a composition is not administered to a subject within about 2 hours prior to surgery.

In some embodiments, a nutritionally-effective dose of a composition can be administered with food. In some embodiments, a nutritionally-effective dose of a composition can be administered without food. In some embodiments, a plurality of nutritionally-effective doses of a composition can be administered with food. In some embodiments, a plurality of nutritionally-effective doses of a composition can be administered without food. In some embodiments, a nutritionally-effective dose of a composition can be administered with food and an additional nutritionally-effective dose of a composition can be administered without food. In some embodiments, a plurality of nutritionally-effective doses of a composition can be administered with food according to the recommendation of a doctor.

The present disclosure provides methods and compositions for administering a nutritionally-effective amount of a pre-surgical nutritional supplement to a subject. Non-limiting examples of surgery include appendectomy, arthroscopy, bariatric surgery, biopsy, breast biopsy, liver biopsy, breast surgery, bronchoscopy, carotid endarterectomy, cataract surgery, cesarean section, cholecystectomy, colorectal surgery, colectomy, coronary artery bypass, cystoscopy, debridement (e.g. wounds, burns, or infections), dental surgery, endocrine surgery, endoscopy, general surgery, graft surgery (e.g., skin grafting), gynecological surgery, hand surgery, head and neck surgery, heart surgery, hemorrhoidectomy, hysterectomy, hysteroscopy, hernia surgery, laparoscopy, laryngoscopy, mastectomy, minimally invasive surgery, neurosurgery, orthopedic surgery, ophthalmological surgery, outpatient surgery pediatric surgery, plastic surgery, prostatectomy, robotic surgery, sigmoidoscopy, spinal surgery, thoracic surgery, thyroid surgery, tonsillectomy, trauma surgery, urologic surgery, and vascular surgery.

In some embodiments, administration of a composition of the disclosure can improve a subject's outcome after surgery. In some embodiments, administration of a composition of the disclosure can decrease a subject's recovery time after surgery. In some embodiments, administration of a composition of the disclosure can decrease a subject's recovery time after surgery compared to a similar subject undergoing the same surgical procedure without administration of a composition of the disclosure. In some embodiments, administration of a composition of the disclose can decrease a subject's recovery time by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to a subject undergoing the same surgical procedure without administration of a composition of the disclosure. In some embodiments, administration of a composition of the disclosure can decrease levels of reactive oxygen species generated in a subject following surgery. In some embodiments, administration of a composition of the disclosure can decrease levels of reactive oxygen species generated in a subject following surgery compared to a similar subject undergoing the same surgical procedure without administration of a composition of the disclosure. In some embodiments, administration of a composition of the disclosure can decrease levels of reactive oxygen species generated in a subject following surgery by least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to a subject undergoing the same surgical procedure without administration of a composition of the disclosure. In some embodiments, levels of reactive oxygen species in a subject are assessed by examining circulating levels of a reactive oxygen species marker in a subject sample (e.g., plasma, or blood) obtained following surgery. In some embodiments, administration of a composition of the disclosure can improve wound healing in a subject following surgery. In some embodiments, administration of a composition of the disclosure can improve wound healing in a subject following surgery compared to a similar subject undergoing the same surgical procedure without administration of a composition of the disclosure. In some embodiments, administration of a composition of the disclosure can improve wound healing in a subject by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to a subject undergoing the same surgical procedure without administration of a composition of the disclosure. In some embodiments, administration of a composition of the disclosure can decrease metabolic needs in a subject following surgery. In some embodiments, administration of a composition of the disclosure can decrease metabolic needs in a subject following surgery compared to a similar subject undergoing the same surgical procedure without administration of a composition of the disclosure. In some embodiments, administration of a composition of the disclosure can decrease metabolic needs in a subject by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to a subject undergoing the same surgical procedure without administration of a composition of the disclosure. In some embodiments, administration of a composition of the disclosure can decrease postoperative complications in a subject following surgery. In some embodiments, administration of a composition of the disclosure can decrease postoperative complications in a subject following surgery compared to a similar subject undergoing the same surgical procedure without administration of a composition of the disclosure. In some embodiments, administration of a composition of the disclosure can decrease postoperative complications in a subject by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% compared to a subject undergoing the same surgical procedure without administration of a composition of the disclosure. Non-limiting examples of postoperative complications include increased risk of sarcopenia, infection, sustained hyperglycemia, organ damage, cognitive impairment, anxiety, reduced cardiac performance, hypertension, post operative nausea and vomiting, impaired wound healing, post operative diabetes, impaired bowel function, and systemic inflammation.

Systems and methods of the present disclosure are advantageous in having a measurable, significant impact on surgical outcomes. Hypoallergenic compositions favorably affect the primary mechanisms of reduced oxidation and tissue degradation caused by anesthetic agents and surgery; enhance immune response; and reduce inflammation, edema and bruising and infection risk. Non-hyperglycemic compositions are beneficial for use by diabetic patients.

In some embodiments, a composition of the disclosure is not administered to a subject with a pre-existing medical condition. In some embodiments, a composition of the disclosure is not administered to a subject with chronic kidney disease. In some embodiments, a composition of the disclosure is not administered to a subject with pre-existing chronic kidney disease stage 1. In some embodiments, a composition of the disclosure is not administered to a subject with pre-existing chronic kidney disease stage 2. In some embodiments, a composition of the disclosure is not administered to a subject with pre-existing chronic kidney disease stage 3A. In some embodiments, a composition of the disclosure is not administered to a subject with pre-existing chronic kidney disease stage 3B. In some embodiments, a composition of the disclosure is not administered to a subject with pre-existing chronic kidney disease stage 4. In some embodiments, a composition of the disclosure is not administered to a subject with pre-existing chronic kidney disease stage 5. In some embodiments, a composition of the disclosure is not administered to a subject with hepatic failure. In some embodiments, a composition of the disclosure is not administered to a subject with hepatic failure stage 1. In some embodiments, a composition of the disclosure is not administered to a subject with hepatic failure stage 2. In some embodiments, a composition of the disclosure is not administered to a subject with hepatic failure stage 3. In some embodiments, a composition of the disclosure is not administered to a subject with hepatic failure stage 4.

In some embodiments, a composition of the disclosure can interact with a concomitant medical therapy. In some embodiments, a composition of the disclosure can interact with an angiotensin-converting enzyme (ACE) inhibitor. In some embodiments, a composition of the disclosure can interact with an angiotensin receptor blocker (ARB). In some embodiments, a composition of the disclosure can interact with a potassium-sparing diuretic. In some embodiments, the potassium-sparing diuretic is amiloride. In some embodiments, the potassium-sparing diuretic is spironolactone. In some embodiments, a composition of the disclosure can interact with a loop diuretic. In some embodiments, the loop diuretic is furosemide. In some embodiments, the loop diuretic is bumetanide. In some embodiments, a composition of the disclosure can interact with a thiazide diuretic. In some embodiments, the thiazide diuretic is chlorothiazide. In some embodiments, the thiazide diuretic is metolazone.

### Kits, Sachets, Bottles, and Compositions

Compositions disclosed herein can be packaged as a kit. Compositions of the invention can be packaged in individual sachets in a kit. Compositions of the invention can be packaged in individual sachets acceptable for a nutritional product. Compositions disclosed herein can be packaged as a potable mixture suitable for human consumption. Compositions disclosed herein can be packaged as a potable mixture suitable for human consumption in a bottle. In some embodiments, a kit or bottle includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label.

### EXAMPLES

### Example 1: Sachet Formulations Comprising Vitamin A, Vitamin C, Vitamin D, Vitamin E, Panthothenic Acid, Calcium, Magnesium, Zinc, Selenomethionine, Copper, Potassium, and Trehalose

A sachet formulation with total mass of 27.6 grams is prepared. TABLES 1A-1D show the components and amounts of example compositions. In some embodiments, the sachet formulation comprises the formulation of TABLE 1A. In some embodiments, the sachet formulation comprises the formulation of TABLE 1B. In some embodiments, the sachet formulation comprises the formulation of TABLE 1C. FIG. 1 illustrates a nutrition label for the formulation of TABLE 1C. In some embodiments, the sachet formulation comprises the formulation of TABLE 1D. A sachet formulation can be divided into two equal doses of 13.8 g each and packaged into individual containers. An individual dose can be dissolved in water and administered to a subject in need of pre-surgical nutritional supplementation at least about 12 hours prior to surgery. At least about 10 hours after the administering, an additional dose can be dissolved in water and administered to a subject in need of pre-surgical nutritional supplementation. At least about 2 hours after the second administering, surgery can be performed on the subject.

TABLE 1A

| Ingredient | Mass (mg) | Weight % (w/w) |
|---|---|---|
| Vitamin A | 0.04 | 0.0001 |
| Vitamin C | 2500 | 9.1 |
| Vitamin D | 0.05 | 0.0002 |
| Vitamin E | 400 | 1.4 |
| Pantothenic Acid | 2000 | 7.2 |
| Calcium | 200 | 0.7 |
| Magnesium | 350 | 1.3 |
| Zinc | 30 | 0.1 |
| Selenomethionine | 0.5 | 0.0020 |
| Copper | 0.9 | 0.0030 |
| Potassium | 250 | 0.9 |
| Trehalose | 14000 | 50.7 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 7868.51 | 28.5 |
| flavorant, sweetener) | | |

TABLE 1B

| Ingredient | Mass (mg) | Weight % (w/w) |
|---|---|---|
| Vitamin A | 0.04 | 0.0001 |
| Vitamin C | 3000 | 10.9 |
| Vitamin D | 0.06 | 0.0002 |
| Vitamin E | 450 | 1.6 |
| Pantothenic Acid | 2250 | 8.2 |
| Calcium | 250 | 0.9 |
| Magnesium | 325 | 1.2 |
| Zinc | 28 | 0.1 |
| Selenomethionine | 0.6 | 0.0020 |
| Copper | 0.8 | 0.0030 |
| Potassium | 325 | 1.2 |
| Trehalose | 15000 | 54.3 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 5970.5 | 21.6 |
| flavorant, sweetener) | | |

TABLE 1C

| Ingredient | Mass (mg) | Weight % (w/w) |
|---|---|---|
| Vitamin A | 0.05 | 0.0002 |
| Vitamin C | 3000 | 10.9 |
| Vitamin D | 0.06 | 0.0002 |
| Vitamin E | 500 | 1.8 |
| Pantothenic Acid | 2200 | 8.0 |
| Calcium | 230 | 0.8 |
| Magnesium | 375 | 1.4 |
| Zinc | 45 | 0.2 |
| Selenomethionine | 0.6 | 0.0020 |
| Copper | 0.8 | 0.0030 |
| Potassium | 300 | 1.1 |
| Trehalose | 14500 | 52.5 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 6448.49 | 23.4 |
| flavorant, sweetener) | | |

TABLE 1D

| Ingredient | Mass (mg) | Weight % (w/w) |
|---|---|---|
| Vitamin A | 0.03 | 0.0001 |
| Vitamin C | 2000 | 7.2 |
| Vitamin D | 0.075 | 0.0003 |
| Vitamin E | 400 | 1.4 |
| Pantothenic Acid | 2250 | 8.2 |
| Calcium | 250 | 0.9 |
| Magnesium | 300 | 1.1 |
| Zinc | 35 | 0.1 |
| Selenomethionine | 0.5 | 0.0020 |
| Copper | 1 | 0.0040 |
| Potassium | 250 | 0.9 |
| Trehalose | 14500 | 52.5 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 7613.395 | 27.6 |
| flavorant, sweetener) | | |

### Example 2: Sachet Formulations Comprising Vitamin A, Vitamin C, Vitamin D, Vitamin E, Pantothenic Acid, Calcium, Magnesium, Zinc, Selenomethionine, Copper, Potassium, Trehalose, and Arabinogalactan

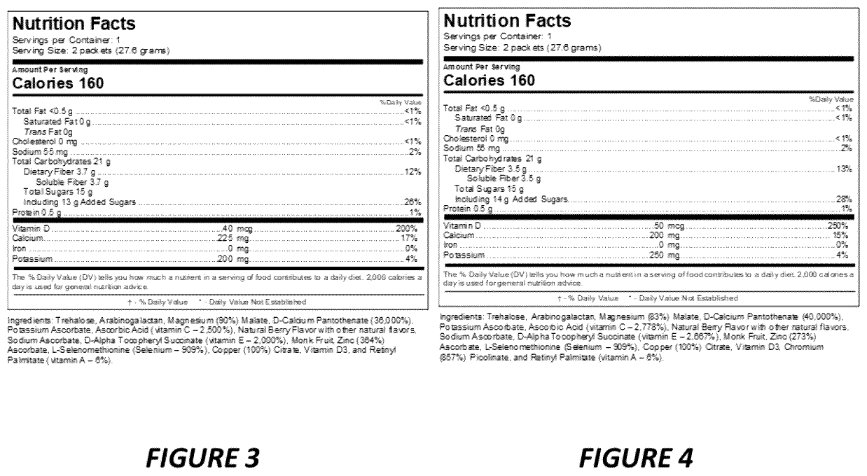

A sachet formulation with total mass of 27.6 grams is prepared. TABLES 2A-2D show the components and amounts of example compositions. In some embodiments, the sachet formulation comprises the formulation of TABLE 2A. FIG. 2 illustrates a nutrition label for a composition of TABLE 2A. In some embodiments, the sachet formulation comprises the formulation of TABLE 2B. In some embodiments, the sachet formulation comprises the formulation of TABLE 2C. In some embodiments, the sachet formulation comprises the formulation of TABLE 2D. FIG. 3 illustrates a nutrition label for a composition of TABLE 2D. A sachet formulation can be divided into two equal doses of 13.8 g each and packaged into individual containers. An individual dose can be dissolved in water and administered to a subject in need of pre-surgical nutritional supplementation at least about 12 hours prior to surgery. At least about 10 hours after the administering, an additional dose can be dissolved in water and administered to a subject in need of pre-surgical nutritional supplementation. At least about 2 hours after the second administering, surgery can be performed on the subject.

TABLE 2A

| Ingredient | Mass (mg) | Weight % (w/w) |
|---|---|---|
| Vitamin A | 0.04 | 0.0001 |
| Vitamin C | 2500 | 9.1 |
| Vitamin D | 0.05 | 0.0002 |
| Vitamin E | 400 | 1.4 |
| Pantothenic Acid | 2000 | 7.2 |
| Calcium | 200 | 0.7 |
| Magnesium | 350 | 1.3 |
| Zinc | 30 | 0.1 |
| Selenomethionine | 0.5 | 0.002 |
| Copper | 0.9 | 0.003 |
| Potassium | 250 | 0.9 |
| Trehalose | 14000 | 50.7 |
| Arabinogalactan | 3500 | 12.7 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 4368.51 | 15.8 |
| flavorant, sweetener) | | |

TABLE 2B

| Ingredient | Mass (mg) | Weight % (w/w) |
|---|---|---|
| Vitamin A | 0.06 | 0.0002 |
| Vitamin C | 2500 | 9.1 |
| Vitamin D | 0.025 | 0.0001 |
| Vitamin E | 500 | 1.8 |
| Pantothenic Acid | 2500 | 9.1 |
| Calcium | 210 | 0.8 |
| Magnesium | 400 | 1.4 |
| Zinc | 25 | 0.1 |
| Selenomethionine | 0.6 | 0.002 |
| Copper | 1 | 0.004 |
| Potassium | 225 | 0.8 |
| Trehalose | 13500 | 48.9 |
| Arabinogalactan | 3000 | 10.9 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 4738.315 | 17.2 |
| flavorant, sweetener) | | |

TABLE 2C

| Ingredient | Mass (mg) | Weight % (w/w) |
|---|---|---|
| Vitamin A | 0.05 | 0.0002 |
| Vitamin C | 2250 | 8.2 |
| Vitamin D | 0.07 | 0.0003 |
| Vitamin E | 350 | 1.3 |
| Pantothenic Acid | 1750 | 6.3 |
| Calcium | 175 | 0.6 |
| Magnesium | 325 | 1.2 |
| Zinc | 35 | 0.1 |
| Selenomethionine | 0.6 | 0.002 |
| Copper | 0.8 | 0.003 |
| Potassium | 300 | 1.1 |
| Trehalose | 14500 | 52.5 |
| Arabinogalactan | 4000 | 14.5 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 3913.48 | 14.2 |
| flavorant, sweetener) | | |

TABLE 2D

| Ingredient | Mass (mg) | Weight % (w/w) |
|---|---|---|
| Vitamin A | 0.05 | 0.0002 |
| Vitamin C | 2250 | 8.2 |
| Vitamin D | 0.04 | 0.0001 |
| Vitamin E | 300 | 1.1 |
| Pantothenic Acid | 1800 | 6.5 |
| Calcium | 225 | 0.8 |
| Magnesium | 375 | 1.4 |
| Zinc | 40 | 0.1 |
| Selenomethionine | 0.5 | 0.002 |
| Copper | 0.9 | 0.003 |
| Potassium | 200 | 0.7 |
| Trehalose | 13000 | 47.1 |
| Arabinogalactan | 3750 | 13.6 |
| L-citrulline | 0 | 0.0 |
| Chromium | 0 | 0.0 |
| Manganese | 0 | 0.0 |
| Carnosine | 0 | 0.0 |
| Gelatin | 0 | 0.0 |
| Other (e.g., excipient, diluent, | 5658.51 | 20.5 |
| flavorant, sweetener) | | |

### Example 3: Sachet Formulations Comprising Vitamin A, Vitamin C, Vitamin D, Vitamin E, Pantothenic Acid, Calcium, Magnesium, Zinc, Selenomethionine, Copper, Potassium, Trehalose, Arabinogalactan, L-Citrulline, and Chromium

A sachet formulation with total mass of 27.6 grams is prepared. TABLES 3A-3D show the components and amounts of example compositions. In some embodiments, the sachet formulation comprises the formulation of TABLE 3A. FIG. 4 illustrates a nutrition label for a composition of TABLE 3A. In some embodiments, the sachet formulation comprises the formulation of TABLE 3B. In some embodiments, the sachet formulation comprises the formulation of TABLE 3C. In some embodiments, the sachet formulation comprises the formulation of TABLE 3D. A sachet formulation can be divided into two equal doses of 13.8 g each and packaged into individual containers. An individual dose can be dissolved in water and administered to a subject in need of pre-surgical nutritional supplementation at least about 12 hours prior to surgery. At least about 10 hours after the administering, an additional dose can be dissolved in water and administered to a subject in need of pre-surgical nutritional supplementation. At least about 2 hours after the second administering, surgery can be performed on the subject.

TABLE 3A

| Ingredient | Mass (mg) | Weight % (w/w) |
|---|---|---|
| Vitamin A | 0.04 | 0.0001 |
| Vitamin C | 2500 | 9.1 |
| Vitamin D | 0.05 | 0.0002 |
| Vitamin E | 400 | 1.4 |
| Pantothenic Acid | 2000 | 7.2 |
| Calcium | 200 | 0.7 |
| Magnesium | 350 | 1.3 |
| Zinc | 30 | 0.1 |
| Selenomethionine | 0.5 | 0.002 |
| Copper | 0.9 | 0.003 |
| Potassium | 250 | 0.9 |
| Trehalose | 14000 | 50.7 |
| Arabinogalactan | 3500 | 12.7 |
| L-citrulline | 3000 | 10.9 |
| Chromium | 300 | 1.1 |
| Manganese | 0 | 0.0 |
| Carnosine | 0 | 0.0 |
| Gelatin | 0 | 0.0 |
| Other (e.g., excipient, diluent, | 1068.51 | 3.9 |
| flavorant, sweetener) | | |

TABLE 3B

| Ingredient | Mass (mg) | Weight % (w/w) |
|---|---|---|
| Vitamin A | 0.04 | 0.0001 |
| Vitamin C | 3000 | 10.9 |
| Vitamin D | 0.07 | 0.0003 |
| Vitamin E | 500 | 1.8 |
| Pantothenic Acid | 2250 | 8.2 |
| Calcium | 210 | 0.8 |
| Magnesium | 400 | 1.4 |
| Zinc | 40 | 0.1 |
| Selenomethionine | 0.6 | 0.002 |
| Copper | 1 | 0.004 |
| Potassium | 300 | 1.1 |
| Trehalose | 13500 | 48.9 |
| Arabinogalactan | 3500 | 12.7 |
| L-citrulline | 3000 | 10.9 |
| Chromium | 250 | 0.9 |
| Manganese | 0 | 0.0 |
| Carnosine | 0 | 0.0 |
| Gelatin | 0 | 0.0 |
| Other (e.g., excipient, diluent, | 648.29 | 2.3 |
| flavorant, sweetener) | | |

TABLE 3C

| Ingredient | Mass (mg) | Weight % (w/w) |
|---|---|---|
| Vitamin A | 0.05 | 0.0002 |
| Vitamin C | 2000 | 7.2 |
| Vitamin D | 0.06 | 0.0002 |
| Vitamin E | 450 | 1.6 |
| Pantothenic Acid | 2500 | 9.1 |
| Calcium | 250 | 0.9 |
| Magnesium | 375 | 1.4 |
| Zinc | 35 | 0.1 |
| Selenomethionine | 0.75 | 0.003 |
| Copper | 0.75 | 0.003 |
| Potassium | 250 | 0.9 |
| Trehalose | 14000 | 50.7 |
| Arabinogalactan | 4000 | 14.5 |
| L-citrulline | 2500 | 9.1 |
| Chromium | 250 | 0.9 |
| Manganese | 0 | 0.0 |
| Carnosine | 0 | 0.0 |
| Gelatin | 0 | 0.0 |
| Other (e.g., excipient, diluent, | 988.39 | 3.6 |
| flavorant, sweetener) | | |

TABLE 3D

| Ingredient | Mass (mg) | Weight % (w/w) |
|---|---|---|
| Vitamin A | 0.04 | 0.0001 |
| Vitamin C | 2750 | 10.0 |
| Vitamin D | 0.05 | 0.0002 |
| Vitamin E | 375 | 1.4 |
| Pantothenic Acid | 1800 | 6.5 |
| Calcium | 175 | 0.6 |
| Magnesium | 425 | 1.5 |
| Zinc | 25 | 0.1 |
| Selenomethionine | 0.7 | 0.003 |
| Copper | 0.7 | 0.003 |
| Potassium | 275 | 1.0 |
| Trehalose | 15000 | 54.3 |
| Arabinogalactan | 3000 | 10.9 |
| L-citrulline | 2500 | 9.1 |
| Chromium | 300 | 1.1 |
| Manganese | 0 | 0.0 |
| Carnosine | 0 | 0.0 |
| Gelatin | 0 | 0.0 |
| Other (e.g., excipient, diluent, | 973.51 | 3.5 |
| flavorant, sweetener) | | |

### Example 4: Sachet Formulations Comprising Vitamin A, Vitamin C, Vitamin D, Vitamin E, Pantothenic Acid, Calcium, Magnesium, Zinc, Selenomethionine, Copper, Potassium, Trehalose, Arabinogalactan, Manganese, and Carnosine

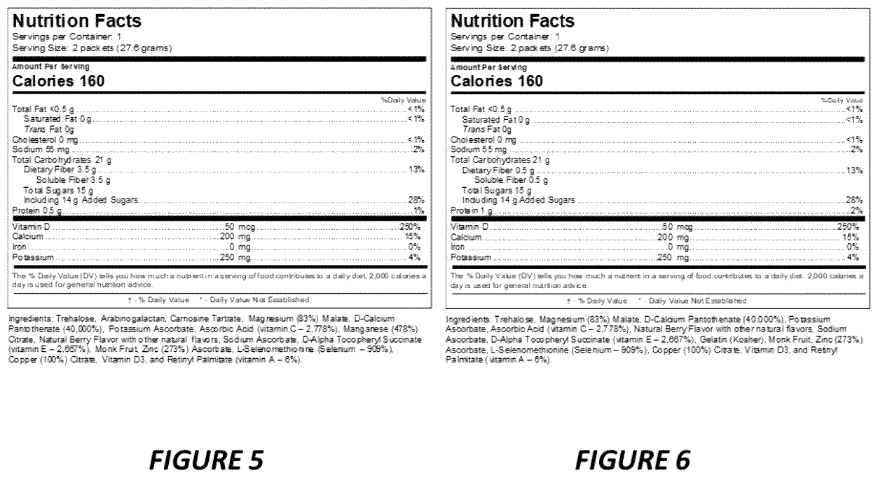

A sachet formulation with total mass of 27.6 grams is prepared. TABLES 4A-4D show the components and amounts of example compositions. In some embodiments, the sachet formulation comprises the formulation of TABLE 4A. FIG. 5 illustrates a nutrition label for a composition of TABLE 4A. In some embodiments, the sachet formulation comprises the formulation of TABLE 4B. In some embodiments, the sachet formulation comprises the formulation of TABLE 4C. In some embodiments, the sachet formulation comprises the formulation of TABLE 4D. A sachet formulation can be divided into two equal doses of 13.8 g each and packaged into individual containers. An individual dose can be dissolved in water and administered to a subject in need of pre-surgical nutritional supplementation at least about 12 hours prior to surgery. At least about 10 hours after the administering, an additional dose can be dissolved in water and administered to a subject in need of pre-surgical nutritional supplementation. At least about 2 hours after the second administering, surgery can be performed on the subject.

TABLE 4A

| Ingredient | Mass (mg) | Weight % (w/w) |
|---|---|---|
| Vitamin A | 0.04 | 0.0001 |
| Vitamin C | 2500 | 9.1 |
| Vitamin D | 0.05 | 0.0002 |
| Vitamin E | 400 | 1.4 |
| Pantothenic Acid | 2000 | 7.2 |
| Calcium | 200 | 0.7 |
| Magnesium | 350 | 1.3 |
| Zinc | 30 | 0.1 |
| Selenomethionine | 0.5 | 0.002 |
| Copper | 0.9 | 0.003 |
| Potassium | 250 | 0.9 |
| Trehalose | 14000 | 50.7 |
| Arabinogalactan | 3500 | 12.7 |
| L-citrulline | 0 | 0.0 |
| Chromium | 0 | 0.0 |
| Manganese | 11 | 0.04 |
| Carnosine | 2000 | 7.2 |
| Gelatin | 0 | 0.0 |
| Other (e.g., excipient, diluent, | 2357.51 | 8.5 |
| flavorant, sweetener) | | |

TABLE 4B

| Ingredient | Mass (mg) | Weight % (w/w) |
|---|---|---|
| Vitamin A | 0.04 | 0.0001 |
| Vitamin C | 2000 | 7.2 |
| Vitamin D | 0.04 | 0.0001 |
| Vitamin E | 400 | 1.4 |
| Pantothenic Acid | 2200 | 8.0 |
| Calcium | 250 | 0.9 |
| Magnesium | 350 | 1.3 |
| Zinc | 25 | 0.1 |
| Selenomethionine | 0.5 | 0.002 |
| Copper | 0.75 | 0.003 |
| Potassium | 250 | 0.9 |
| Trehalose | 14000 | 50.7 |
| Arabinogalactan | 3500 | 12.7 |
| L-citrulline | 0 | 0.0 |
| Chromium | 0 | 0.0 |
| Manganese | 9 | 0.03 |
| Carnosine | 1500 | 5.4 |
| Gelatin | 0 | 0.0 |
| Other (e.g., excipient, diluent, | 3108.67 | 11.3 |
| flavorant, sweetener) | | |

TABLE 4C

| Ingredient | Mass (mg) | Weight % (w/w) |
|---|---|---|
| Vitamin A | 0.05 | 0.0002 |
| Vitamin C | 2000 | 7.2 |
| Vitamin D | 0.06 | 0.0002 |
| Vitamin E | 450 | 1.6 |
| Pantothenic Acid | 2500 | 9.1 |
| Calcium | 150 | 0.5 |
| Magnesium | 410 | 1.5 |
| Zinc | 35 | 0.1 |
| Selenomethionine | 0.5 | 0.002 |
| Copper | 1 | 0.004 |
| Potassium | 300 | 1.1 |
| Trehalose | 14500 | 52.5 |
| Arabinogalactan | 3000 | 10.9 |
| L-citrulline | 0 | 0.0 |
| Chromium | 0 | 0.0 |
| Manganese | 10 | 0.03 |
| Carnosine | 1750 | 6.3 |
| Gelatin | 0 | 0.0 |
| Other (e.g., excipient, diluent, | 2490.84 | 9.0 |
| flavorant, sweetener) | | |

TABLE 4D

| Ingredient | Mass (mg) | Weight % (w/w) |
|---|---|---|
| Vitamin A | 0.03 | 0.0001 |
| Vitamin C | 3000 | 10.9 |
| Vitamin D | 0.05 | 0.0002 |
| Vitamin E | 425 | 1.5 |
| Pantothenic Acid | 2200 | 8.0 |
| Calcium | 200 | 0.7 |
| Magnesium | 360 | 1.3 |
| Zinc | 40 | 0.1 |
| Selenomethionine | 0.35 | 0.001 |
| Copper | 0.75 | 0.003 |
| Potassium | 250 | 0.9 |
| Trehalose | 15000 | 54.3 |
| Arabinogalactan | 2750 | 10.0 |
| L-citrulline | 0 | 0.0 |
| Chromium | 0 | 0.0 |
| Manganese | 7.5 | 0.03 |
| Carnosine | 2500 | 9.1 |
| Gelatin | 0 | 0.0 |
| Other (e.g., excipient, diluent, | 866.32 | 3.1 |
| flavorant, sweetener) | | |

### Example 5: Sachet Formulations Comprising Vitamin A, Vitamin C, Vitamin D, Vitamin E, Pantothenic Acid, Calcium, Magnesium, Zinc, Selenomethionine, Copper, Potassium, Trehalose, and Gelatin

A sachet formulation with total mass of 27.6 grams is prepared. TABLES 5A-5D show the components and amounts of example compositions. In some embodiments, the sachet formulation comprises the formulation of TABLE 5A. FIG. 6 illustrates a nutrition label for a composition of TABLE 5A. In some embodiments, the sachet formulation comprises the formulation of TABLE 5B. In some embodiments, the sachet formulation comprises the formulation of TABLE 5C. In some embodiments, the sachet formulation comprises the formulation of TABLE 5D. A sachet formulation can be divided into two equal doses of 13.8 g each and packaged into individual containers. An individual dose can be dissolved in water and administered to a subject in need of pre-surgical nutritional supplementation at least about 12 hours prior to surgery. At least about 10 hours after the administering, an additional dose can be dissolved in water and administered to a subject in need of pre-surgical nutritional supplementation. At least about 2 hours after the second administering, surgery can be performed on the subject.

TABLE 5A

| Ingredient | Mass (mg) | Weight % (w/w) |
|---|---|---|
| Vitamin A | 0.04 | 0.0001 |
| Vitamin C | 2500 | 9.1 |
| Vitamin D | 0.05 | 0.0002 |
| Vitamin E | 400 | 1.4 |
| Pantothenic Acid | 2000 | 7.2 |
| Calcium | 200 | 0.7 |
| Magnesium | 350 | 1.3 |
| Zinc | 30 | 0.1 |
| Selenomethionine | 0.5 | 0.002 |
| Copper | 0.9 | 0.003 |
| Potassium | 250 | 0.9 |
| Trehalose | 14000 | 50.7 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 350 | 1.3 |
| Other (e.g., excipient, diluent, | 7518.51 | 27.2 |
| flavorant, sweetener) | | |

TABLE 5B

| Ingredient | Mass (mg) | Weight % (w/w) |
|---|---|---|
| Vitamin A | 0.05 | 0.0002 |
| Vitamin C | 2500 | 9.1 |
| Vitamin D | 0.4 | 0.001 |
| Vitamin E | 300 | 1.1 |
| Pantothenic Acid | 2500 | 9.1 |
| Calcium | 150 | 0.5 |
| Magnesium | 250 | 0.9 |
| Zinc | 15 | 0.1 |
| Selenomethionine | 0.35 | 0.001 |
| Copper | 0.5 | 0.002 |
| Potassium | 175 | 0.6 |
| Trehalose | 14500 | 52.5 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 500 | 1.8 |
| Other (e.g., excipient, diluent, | 6708.7 | 24.3 |
| flavorant, sweetener) | | |

TABLE 5C

| Nutrient | Mass (mg) | Weight % (w/w) |
|---|---|---|
| Vitamin A | 0.035 | 0.0001 |
| Vitamin C | 2250 | 8.2 |
| Vitamin D | 0.05 | 0.0002 |
| Vitamin E | 450 | 1.6 |
| Pantothenic Acid | 2000 | 7.2 |
| Calcium | 200 | 0.7 |
| Magnesium | 350 | 1.3 |
| Zinc | 25 | 0.1 |
| Selenomethionine | 0.4 | 0.001 |
| Copper | 0.8 | 0.003 |
| Potassium | 300 | 1.1 |
| Trehalose | 15000 | 54.3 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 400 | 1.4 |
| Other (e.g., excipient, diluent, | 6623.715 | 24.0 |
| flavorant, sweetener) | | |

TABLE 5D

| Ingredient | Mass (mg) | Weight % (w/w) |
|---|---|---|
| Vitamin A | 0.04 | 0.0001 |
| Vitamin C | 1850 | 6.7 |
| Vitamin D | 0.06 | 0.0002 |
| Vitamin E | 500 | 1.8 |
| Pantothenic Acid | 2500 | 9.1 |
| Calcium | 150 | 0.5 |
| Magnesium | 300 | 1.1 |
| Zinc | 40 | 0.1 |
| Selenomethionine | 0.55 | 0.002 |
| Copper | 0.8 | 0.003 |
| Potassium | 250 | 0.9 |
| Trehalose | 14250 | 51.6 |
| Arabinogalactan | 0 | 0.0 |
| L-citrulline | 0 | 0.0 |
| Chromium | 0 | 0.0 |
| Manganese | 0 | 0.0 |
| Carnosine | 0 | 0.0 |
| Gelatin | 300 | 1.1 |
| Other (e.g., excipient, diluent, | 7458.55 | 27.0 |
| flavorant, sweetener) | | |

### Example 6: Sachet Manufacturing

A sachet comprising a composition of the disclosure can be prepared and manufactured according to the following procedure:
i) Gathering of individual ingredients
ii) Weighing of individual ingredients
iii) Pre-blending of ingredients that are under 1% by weight
iv) Screening of the pre-blended and all other ingredients through a mesh screen strainer (e.g., 12 mesh to 20 mesh)
v) Combining the pre-blended ingredients and remaining ingredients and blending for in a planetary or v-blender (e.g., for 19 minutes) to form a blended composition
vi) Preparing of nutritional packaging film suitable for human consumption (e.g., transporting and unwinding, printing, film tension and cutting)
vii) Forming a stick pack center seal pillow pouch (e.g., 2″×4.5″ white mylar foil)
viii) Filling the stick pack with 13.8 g of a blended composition per pack with a filling machine (e.g., a custom vertical FFS filling machine)
ix) Sealing the end of a stick pack (e.g., with a heat sealer)
x) Discharging 2 stick packs into a product box
xi) Labeling and packaging the product boxes.

### Example 7: Preparation of a Liquid Composition

A liquid comprising a composition of the disclosure can be prepared and manufactured according to the following procedure:
i) Gathering of individual ingredients
ii) Weighing of individual ingredients
iii) Pre-blending of ingredients that are under 1% by weight
iv) Screening of the pre-blended and all other ingredients through a mesh screen strainer (e.g., 12 mesh to 20 mesh)
v) Combining the pre-blended ingredients and remaining ingredients and blending for in a planetary or v-blender (e.g., for 19 minutes) to form a blended composition
vi) Combining the pre-blended composition with ingredients suitable for shelf stability (e.g., preservatives, solubilizing agent, emulsifying agent) to provide a shelf-stable composition
vi) Combining the shelf-stable composition with a solvent suitable for human consumption to form a liquid composition
vii) Packaging the liquid composition in packaging suitable for human consumption

### Example 8: Administration of a Composition to a Subject

A nutritionally-effective amount of a composition can be administered to a subject in need of pre-surgical nutritional supplementation. A first dose of a composition can be prepared by dissolving one 13.8 g sachet in 8 ounces of water and the resulting mixture stirred until the composition is completely dissolved in the water to yield a dissolved composition. Following the dissolving, the dissolved composition is administered to the subject 12 hours prior to surgery. The subject then waits for at least 10 hours following the first administration of the composition. At least two hours prior to surgery, a second dose of the composition is prepared in the same manner as the first and administered to the subject in a second administration. Two hours after the second administration, the subject undergoes surgery.

A nutritionally-effective amount of a composition can be administered to a subject in need of pre-surgical nutritional supplementation. A first dose of a composition can be administered by providing a pre-made liquid composition to the subject. The subject consumers the pre-made liquid composition 12 hours prior to surgery in a first administration. The subject then waits for at least 10 hours following the first administration of the composition. At least two hours prior to surgery, a second dose of the composition is administered in the same manner as the first and administered to the subject in a second administration. Two hours after the second administration, the subject undergoes surgery.

Possible interactions with concomitant medical therapies include interactions with angiotensin-converting enzyme (ACE) inhibitors; angiotensin receptor blockers (ARBs); potassium-sparing diuretics, including amiloride (Midamor®) and spironolactone (Aldactone®); loop diuretics, including furosemide (Lasix®) and bumetanide (Bumex®); and thiazide diuretics, including chlorothiazide (Diuril®) and metolazone.

Exclusion criteria for administration include the following:
1. Adults with pre-existing Chronic Kidney Disease stage 1 and over.
2. Adults with pre-existing hepatic failure stage 1 and over.

### EMBODIMENTS

Embodiment 1. A composition comprising ingredients, wherein the ingredients comprise, by percent of total mass of the ingredients: i) about 0.00005% w/w to about 0.001% w/w Vitamin A; ii) about 5% w/w to about 15% w/w Vitamin C; iii) about 0.0001% w/w to about 0.01% w/w Vitamin D; iv) about 0.1% w/w to about 10% w/w Vitamin E; v) about 5% to about 15% w/w pantothenic acid; vi) about 0.1% w/w to about 10% w/w calcium; vii) about 0.1% w/w to about 10% w/w magnesium; viii) about 0.01% to about 1% w/w zinc; ix) about 0.001% w/w to about 0.01% w/w selenomethionine; x) about 0.001% w/w to about 0.01% w/w copper; xi) about 0.1% w/w to about 10% w/w potassium; and xii) about 25% w/w to about 75% w/w trehalose.

Embodiment 2. The composition of embodiment 1, the ingredients further comprising xiii) about 5% w/w to about 25% w/w arabinogalactan.

Embodiment 3. The composition of embodiment 1, the ingredients further comprising: xiii) about 5% w/w to about 25% w/w arabinogalactan; xiv) about 5% w/w to about 25% w/w L-citrulline; xv) about 0.1% w/w to about 10% w/w chromium; xvi) about 0.001% w/w to about 0.1% w/w manganese; xvii) about 5% w/w to about 15% w/w carnosine; and xviii) about 0.1% w/w to about 10% w/w gelatin.

Embodiment 4. The composition of embodiment 1, the ingredients comprising: i) about 0.0001% w/w Vitamin A; ii) about 9% w/w Vitamin C; iii) about 0.002% w/w Vitamin D; iv) about 1.5% w/w Vitamin E; v) about 7% w/w pantothenic acid; vi) about 1% w/w calcium; vii) about 1% w/w magnesium; viii) about 0.1% w/w zinc; ix) about 0.002% w/w selenomethionine; x) about 0.003% w/w copper; xi) about 0.9% w/w potassium; and xii) about 51% w/w trehalose.

Embodiment 5. The composition of any one of embodiments 3-4, the ingredients comprising xiii) about 13% w/w arabinogalactan.

Embodiment 6. The composition of embodiment 3, the ingredients comprising: xiii) about 13% w/w arabinogalactan; xiv) about 11% w/w L-citrulline; xv) about 1% w/w chromium; xvi) about 0.04% w/w manganese; xvii) about 7% w/w carnosine; and xviii) about 1% w/w gelatin.

Embodiment 7. The composition of any one of embodiments 1-6, further comprising a solvent, wherein the ingredients are mixed into the solvent to provide a potable mixture that is suitable for human consumption.

Embodiment 8. The composition of embodiment 7, wherein the solvent is water.

Embodiment 9. The composition of any one of embodiments 1-8, wherein the composition is hypoallergenic.

Embodiment 10. A method comprising contacting a composition of any one of embodiments 1-9 with a diluent to provide a mixture.

Embodiment 11. The method of embodiment 10, further comprising mixing the mixture to obtain in the mixture a consistency that is suitable for human consumption.

Embodiment 12. The method of any one of embodiments 10-11, further comprising packaging the mixture in packaging acceptable for a nutritional product.

Embodiment 13. The method of any one of embodiments 10-12, further comprising packaging the mixture as a sachet acceptable for a nutritional product.

Embodiment 14. A method comprising contacting a composition of any one of embodiments 1-9 with a diluent to provide a solid mixture.

Embodiment 15. The method of embodiment 14, further comprising mixing the solid mixture to obtain in the solid mixture a consistency that is suitable for human consumption.

Embodiment 16. The method of any one of embodiments 14-15, further comprising packaging the solid mixture in packaging acceptable for a nutritional product.

Embodiment 17. The method of any one of embodiments 14-16, further comprising packaging the solid mixture as a sachet acceptable for a nutritional product.

Embodiment 18. A method comprising administering to a subject in need of pre-surgical nutritional supplementation a nutritionally-effective amount of the composition of any of embodiments 1-9.

Embodiment 19. The method of embodiment 18, further comprising performing surgery on the subject after the administering.

Embodiment 20. The method of embodiment 18, further comprising performing surgery on the subject at least about 2 hours after the administering.

Embodiment 21. The method of embodiment 18, further comprising performing surgery on the subject at least about 12 hours after the administering.

Embodiment 22. The method of embodiment 18, further comprising performing surgery on the subject a day after the administering.

Embodiment 23. The method of any one of embodiments 18-22, further comprising providing food to the subject with the administering.

Embodiment 24. The method of any one of embodiments 18-23, further comprising, at least about 10 hours after the administering, performing a second administering to the subject of the composition of any of embodiments 1-9.

Embodiment 25. The method of embodiment 24, further comprising performing surgery on the subject at least about 2 hours after the second administering.

Embodiment 26. A method comprising: a) contacting a composition of any one of embodiments 1-9 with a diluent to provide a mixture; b) mixing the mixture to obtain in the mixture a consistency that is suitable for human consumption; c) administering to a subject in need of pre-surgical nutritional supplementation a nutritionally-effective amount of the mixture; and d) performing surgery on the subject after the administering.

Embodiment 27. The method of embodiment 26, further comprising providing food to the subject with the administering.

Embodiment 28. The method of any one of embodiments 26-27, wherein the performing surgery on the subject is about 2 hours after the administering.

Embodiment 29. The method of any one of embodiments 26-27, wherein the performing surgery on the subject is about 12 hours after the administering.

Embodiment 30. The method of any one of embodiments 26-27, wherein the performing surgery on the subject is a day after the administering.

Embodiment 31. The method of any one of embodiments 26-30, further comprising, after the administering the mixture and prior to the performing surgery, preparing a second mixture, wherein the second mixture is substantially similar to the mixture; mixing the second mixture to obtain in the second mixture a consistency that is suitable for human consumption; and administering to the subject a nutritionally-effective amount of the second mixture.

Embodiment 32. The method of embodiment 31, wherein the administering the second mixture occurs about ten hours after the administering the mixture and about two hours prior to the performing surgery.

Embodiment 33. An article comprising: a) the composition of any one of embodiments 1-9; and b) packaging acceptable for a nutritional product, wherein the composition is disposed inside the packaging.

Embodiment 34. The article of embodiment 33, further comprising written instructions, wherein the written instructions describe use of the composition in the method of any one of embodiments 10-32.

Embodiment 35. The article of embodiment 33, wherein the article is formulated as a sachet.

Embodiment 36. The article of embodiment 33, wherein the article is formulated for reconstitution prior to consumption.

Embodiment 37. A composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; and xii) 14,000 mg trehalose.

Embodiment 38. A composition comprising: i) 40 μg RAE Vitamin A; ii) 3,000 mg Vitamin C; iii) 60 μg Vitamin D; iv) 450 mg Vitamin E; v) 2,250 mg pantothenic acid; vi) 250 mg calcium; vii) 325 mg magnesium; viii) 28 mg zinc; ix) 600 μg selenomethionine; x) 0.8 mg copper; xi) 325 mg potassium; and xii) 15,000 mg trehalose.

Embodiment 39. A composition comprising: i) 30 μg RAE Vitamin A; ii) 2,000 mg Vitamin C; iii) 75 μg Vitamin D; iv) 400 mg Vitamin E; v) 2,250 mg pantothenic acid; vi) 250 mg calcium; vii) 300 mg magnesium; viii) 35 mg zinc; ix) 500 μg selenomethionine; x) 1 mg copper; xi) 250 mg potassium; and xii) 14,500 mg trehalose.

Embodiment 40. A composition comprising: i) 50 μg RAE Vitamin A; ii) 3,000 mg Vitamin C; iii) 60 μg Vitamin D; iv) 500 mg Vitamin E; v) 2,200 mg pantothenic acid; vi) 230 mg calcium; vii) 375 mg magnesium; viii) 45 mg zinc; ix) 600 μg selenomethionine; x) 0.8 mg copper; xi) 300 mg potassium; and xii) 14,500 mg trehalose.

Embodiment 41. A composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; and xiii) 3,500 mg arabinogalactan.

Embodiment 42. A composition comprising: i) 60 μg RAE Vitamin A; ii) 2,500 mg Vitamin C; iii) 25 μg Vitamin D; iv) 500 mg Vitamin E; v) 2,500 mg pantothenic acid; vi) 210 mg calcium; vii) 400 mg magnesium; viii) 25 mg zinc; ix) 600 μg selenomethionine; x) 1 mg copper; xi) 225 mg potassium; xii) 13,500 mg trehalose; and xiii) 3,000 mg arabinogalactan.

Embodiment 43. A composition comprising: i) 50 μg RAE Vitamin A; ii) 2,250 mg Vitamin C; iii) 40 μg Vitamin D; iv) 300 mg Vitamin E; v) 1,800 mg pantothenic acid; vi) 225 mg calcium; vii) 375 mg magnesium; viii) 40 mg zinc; ix) 500 μg selenomethionine; x) 0.9 mg copper; xi) 200 mg potassium; xii) 13,000 mg trehalose; and xiii) 3,750 mg arabinogalactan.

Embodiment 44. A composition comprising: i) 50 μg RAE Vitamin A; ii) 2,250 mg Vitamin C; iii) 70 μg Vitamin D; iv) 350 mg Vitamin E; v) 1,750 mg pantothenic acid; vi) 175 mg calcium; vii) 325 mg magnesium; viii) 35 mg zinc; ix) 600 μg selenomethionine; x) 0.8 mg copper; xi) 300 mg potassium; xii) 14,500 mg trehalose; and xiii) 4,000 mg arabinogalactan.

Embodiment 45. A composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; xiii) 3,500 mg arabinogalactan; xiv) 3,000 mg L-citrulline; and xv) 300 mg chromium.

Embodiment 46. A composition comprising: i) 40 μg RAE Vitamin A; ii) 3,000 mg Vitamin C; iii) 70 μg Vitamin D; iv) 500 mg Vitamin E; v) 2,250 mg pantothenic acid; vi) 210 mg calcium; vii) 400 mg magnesium; viii) 40 mg zinc; ix) 600 μg selenomethionine; x) 1 mg copper; xi) 300 mg potassium; xii) 13,500 mg trehalose; xiii) 3,500 mg arabinogalactan; xiv) 3,000 mg L-citrulline; and xv) 250 mg chromium.

Embodiment 47. A composition comprising: i) 50 μg RAE Vitamin A; ii) 2,000 mg Vitamin C; iii) 60 μg Vitamin D; iv) 450 mg Vitamin E; v) 2,500 mg pantothenic acid; vi) 250 mg calcium; vii) 375 mg magnesium; viii) 35 mg zinc; ix) 750 μg selenomethionine; x) 0.75 mg copper; xi) 250 mg potassium; xii) 14,000 mg trehalose; xiii) 4,000 mg arabinogalactan; xiv) 2,500 mg L-citrulline; and xv) 250 mg chromium.

Embodiment 48. A composition comprising: i) 40 μg RAE Vitamin A; ii) 2,750 mg Vitamin C; iii) 50 μg Vitamin D; iv) 375 mg Vitamin E; v) 1,800 mg pantothenic acid; vi) 175 mg calcium; vii) 425 mg magnesium; viii) 25 mg zinc; ix) 700 μg selenomethionine; x) 0.7 mg copper; xi) 275 mg potassium; xii) 15,000 mg trehalose; xiii) 3,000 mg arabinogalactan; xiv) 2,500 mg L-citrulline; and xv) 300 mg chromium.

Embodiment 49. A composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; xiii) 3,500 mg arabinogalactan; xiv) 11 mg manganese; and xv) 2,000 mg carnosine.

Embodiment 50. A composition comprising: i) 40 μg RAE Vitamin A; ii) 2,000 mg Vitamin C; iii) 40 μg Vitamin D; iv) 400 mg Vitamin E; v) 2,200 mg pantothenic acid; vi) 250 mg calcium; vii) 350 mg magnesium; viii) 25 mg zinc; ix) 500 μg selenomethionine; x) 0.75 mg copper; xi) 250 mg potassium; xii) 14,000 mg trehalose; xiii) 3,500 mg arabinogalactan; xiv) 9 mg manganese; and xv) 1,500 mg carnosine.

Embodiment 51. A composition comprising: i) 50 μg RAE Vitamin A; ii) 2,000 mg Vitamin C; iii) 60 μg Vitamin D; iv) 450 mg Vitamin E; v) 2,500 mg pantothenic acid; vi) 150 mg calcium; vii) 410 mg magnesium; viii) 35 mg zinc; ix) 500 μg selenomethionine; x) 1 mg copper; xi) 300 mg potassium; xii) 14,500 mg trehalose; xiii) 3,000 mg arabinogalactan; xiv) 10 mg manganese; and xv) 1,750 mg carnosine.

Embodiment 52. A composition comprising: i) 30 μg RAE Vitamin A; ii) 3,000 mg Vitamin C; iii) 50 μg Vitamin D; iv) 425 mg Vitamin E; v) 2,200 mg pantothenic acid; vi) 200 mg calcium; vii) 360 mg magnesium; viii) 40 mg zinc; ix) 350 μg selenomethionine; x) 0.75 mg copper; xi) 250 mg potassium; xii) 15,000 mg trehalose; xiii) 2,750 mg arabinogalactan; xiv) 7.5 mg manganese; and xv) 2,500 mg carnosine.

Embodiment 53. A composition comprising: i) 40 μg RAE Vitamin A; ii) 2,500 mg Vitamin C; iii) 50 μg Vitamin D; iv) 400 mg Vitamin E; v) 2,000 mg pantothenic acid; vi) 200 mg calcium; vii) 350 mg magnesium; viii) 30 mg zinc; ix) 500 μg selenomethionine; x) 0.9 mg copper; xi) 250 mg potassium; xii) 14,000 mg trehalose; and xiii) 350 mg gelatin.

Embodiment 54. A composition comprising: i) 50 μg RAE Vitamin A; ii) 2,500 mg Vitamin C; iii) 40 μg Vitamin D; iv) 300 mg Vitamin E; v) 2,500 mg pantothenic acid; vi) 150 mg calcium; vii) 250 mg magnesium; viii) 15 mg zinc; ix) 350 μg selenomethionine; x) 0.5 mg copper; xi) 175 mg potassium; xii) 14,500 mg trehalose; and xiii) 500 mg gelatin.

Embodiment 55. A composition comprising: i) 35 μg RAE Vitamin A; ii) 2,250 mg Vitamin C; iii) 50 μg Vitamin D; iv) 450 mg Vitamin E; v) 2,000 mg pantothenic acid; vi) 200 mg calcium; vii) 350 mg magnesium; viii) 25 mg zinc; ix) 400 μg selenomethionine; x) 0.8 mg copper; xi) 300 mg potassium; xii) 15,000 mg trehalose; and xiii) 400 mg gelatin.

Embodiment 56. A composition comprising: i) 40 μg RAE Vitamin A; ii) 1,850 mg Vitamin C; iii) 60 μg Vitamin D; iv) 500 mg Vitamin E; v) 2,500 mg pantothenic acid; vi) 150 mg calcium; vii) 300 mg magnesium; viii) 40 mg zinc; ix) 550 μg selenomethionine; x) 0.8 mg copper; xi) 250 mg potassium; xii) 14,250 mg trehalose; and xiii) 300 mg gelatin.

Embodiment 57. The compositions according to any one of embodiments 37-56, further comprising a pharmaceutically acceptable adjuvant, diluent, excipient, carrier, additional medicaments, or a combination thereof.

Embodiment 58. A method of providing nutrition to a patient prior to surgery, the method comprising: a) preparing a composition including trehalose, arabinogalactan, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, retinyl palmitate, chromium picolinate, and L-citrulline; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

Embodiment 59. A method of providing nutrition to a patient prior to surgery, the method comprising: a) preparing a composition including trehalose, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, gelatin and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

Embodiment 60. A method of providing nutrition to a patient prior to surgery, the method comprising: a) preparing a composition including trehalose, arabinogalactan, carnosine, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, manganese, and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

Embodiment 61. A method of providing nutrition to a patient prior to surgery, the method comprising: a) preparing a composition comprising trehalose, arabinogalactan, magnesium malate, D-calcium pantothenate, potassium ascorbate, ascorbic acid, sodium ascorbate, D-alpha tocopheryl succinate, zinc ascorbate, L-selenomethionine, copper citrate, vitamin D3, and retinyl palmitate; b) administering a first dose of the composition to the patient about 1 day prior to a surgical procedure; and c) administering a second dose of the composition to the patient prior to the surgical procedure, wherein the second dose is administered after the first dose and not within 2 hours of the surgical procedure.

1. A composition comprising ingredients, wherein the ingredients comprise, by percent of total mass of the ingredients: i) about 0.00005% w/w to about 0.001% w/w Vitamin A; ii) about 5% w/w to about 15% w/w Vitamin C; iii) about 0.0001% w/w to about 0.01% w/w Vitamin D; iv) about 0.1% w/w to about 10% w/w Vitamin E; v) about 5% to about 15% w/w pantothenic acid; vi) about 0.1% w/w to about 10% w/w calcium; vii) about 0.1% w/w to about 10% w/w magnesium; viii) about 0.01% to about 1% w/w zinc; ix) about 0.001% w/w to about 0.01% w/w selenomethionine; x) about 0.001% w/w to about 0.01% w/w copper; xi) about 0.1% w/w to about 10% w/w potassium; and xii) about 25% w/w to about 75% w/w trehalose.

2. The composition of claim 1, the ingredients further comprising: xiii) about 5% w/w to about 25% w/w arabinogalactan.

3. The composition of claim 1, the ingredients further comprising: xiii) about 5% w/w to about 25% w/w arabinogalactan; xiv) about 5% w/w to about 25% w/w L-citrulline; xv) about 0.1% w/w to about 10% w/w chromium; xvi) about 0.001% w/w to about 0.1% w/w manganese; xvii) about 5% w/w to about 15% w/w carnosine; and xviii) about 0.1% w/w to about 10% w/w gelatin.

4. The composition of claim 1, the ingredients comprising: i) about 0.0001% w/w Vitamin A; ii) about 9% w/w Vitamin C; iii) about 0.002% w/w Vitamin D; iv) about 1.5% w/w Vitamin E; v) about 7% w/w pantothenic acid; vi) about 1% w/w calcium; vii) about 1% w/w magnesium; viii) about 0.1% w/w zinc; ix) about 0.002% w/w selenomethionine; x) about 0.003% w/w copper; xi) about 0.9% w/w potassium; and xii) about 51% w/w trehalose.

5. The composition of claim 3, the ingredients comprising xiii) about 13% w/w arabinogalactan.

6. The composition of claim 3, the ingredients comprising: xiii) about 13% w/w arabinogalactan; xiv) about 11% w/w L-citrulline; xv) about 1% w/w chromium; xvi) about 0.04% w/w manganese; xvii) about 7% w/w carnosine; and xviii) about 1% w/w gelatin.

7. The composition of claim 1, further comprising a solvent, wherein the ingredients are mixed into the solvent to provide a potable mixture that is suitable for human consumption.

8. The composition of claim 7, wherein the solvent is water.

9. The composition of claim 1, wherein the composition is hypoallergenic.

10-32. (canceled)

33. An article comprising: a) a composition; and b) packaging acceptable for a nutritional product, wherein the composition is disposed inside the packaging, wherein the composition comprises ingredients, wherein the ingredients comprise, by percent of total mass of the ingredients: i) about 0.00005% w/w to about 0.001% w/w Vitamin A; ii) about 5% w/w to about 15% w/w Vitamin C; iii) about 0.0001% w/w to about 0.01% w/w Vitamin D; iv) about 0.1% w/w to about 10% w/w Vitamin E; v) about 5% to about 15% w/w pantothenic acid; vi) about 0.1% w/w to about 10% w/w calcium; vii) about 0.1% w/w to about 10% w/w magnesium; viii) about 0.01% to about 1% w/w zinc; ix) about 0.001% w/w to about 0.01% w/w selenomethionine; x) about 0.001% w/w to about 0.01% w/w copper; xi) about 0.1% w/w to about 10% w/w potassium; and xii) about 25% w/w to about 75% w/w trehalose.

34.-36. (canceled)

37. The article of claim 33, the ingredients further comprising about 5% w/w to about 25% w/w arabinogalactan.

38. The article of claim 33, the ingredients further comprising: xiii) about 5% w/w to about 25% w/w arabinogalactan; xiv) about 5% w/w to about 25% w/w L-citrulline; xv) about 0.1% w/w to about 10% w/w chromium; xvi) about 0.001% w/w to about 0.1% w/w manganese; xvii) about 5% w/w to about 15% w/w carnosine; and xviii) about 0.1% w/w to about 10% w/w gelatin.

39. The article of claim 33, the ingredients comprising: i) about 0.0001% w/w Vitamin A; ii) about 9% w/w Vitamin C; iii) about 0.002% w/w Vitamin D; iv) about 1.5% w/w Vitamin E; v) about 7% w/w pantothenic acid; vi) about 1% w/w calcium; vii) about 1% w/w magnesium; viii) about 0.1% w/w zinc; ix) about 0.002% w/w selenomethionine; x) about 0.003% w/w copper; xi) about 0.9% w/w potassium; and xii) about 51% w/w trehalose.

40. The article of claim 38, the ingredients comprising xiii) about 13% w/w arabinogalactan.

41. The article of claim 38, the ingredients comprising: xiii) about 13% w/w arabinogalactan; xiv) about 11% w/w L-citrulline; xv) about 1% w/w chromium; xvi) about 0.04% w/w manganese; xvii) about 7% w/w carnosine; and xviii) about 1% w/w gelatin.

42. The article of claim 33, further comprising a solvent, wherein the ingredients are mixed into the solvent to provide a potable mixture that is suitable for human consumption.

43. The article of claim 42, wherein the solvent is water.

44. The article of claim 33, wherein the composition is hypoallergenic.

45. The article of claim 33, wherein the article is formulated as a sachet.

46. The article of claim 33, wherein the article is formulated for reconstitution prior to consumption.
